(19) United States
(12) Patent Application Publication
(10) Pub. No.: US 2023/0157858 A1
(43) Pub. Date: May 25, 2023

(54) Scoliosis Brace

(71) Applicant: Vincent A. Benenati, Dix Hills, NY (US)

(72) Inventors: Vincent A. Benenati, Dix Hills, NY (US); Diogo Roquette Osorio, East Stroudsburg, PA (US)

(73) Assignee: Vincent A. Benenati, Dix Hills, NY (US)

(21) Appl. No.: 18/071,616

(22) Filed: Nov. 29, 2022

Related U.S. Application Data
(63) Continuation-in-part of application No. 16/926,705, filed on Jul. 12, 2020, now Pat. No. 11,510,802.
(60) Provisional application No. 62/873,845, filed on Jul. 12, 2019.
(60) Provisional application No. 62/873,846, filed on Jul. 13, 2019.

(51) Int. Cl. A61F 5/02

(52) U.S. Cl. CPC A61F 5/024; A61F 5/028; A61F 2005/0167

(57) ABSTRACT

A scoliosis support system designed to relieve pain and improve spinal balance in scoliosis patients with different curve types is disclosed as a tension-based scoliosis orthosis, known as a scoliosis brace or bracing system, that comprises a back panel having a Y shape formed of three sections and a central portion, each section having a rigid de-rotational pad at the section's distal end, and the sections are joined to and/or at the central portion. This scoliosis brace includes inelastic straps adapted to be connected to the de-rotational pads and to be attached to a front of a lumbar belt adapted to be attached to the central portion of the back panel. The bracing system is adapted for multiple types and degrees of adjustment to accommodate different sizes and deformations, adapting the brace to be fitted and adjusted to multiple scoliosis curve types and patient characteristics.

### CROSS REFERENCE TO RELATED APPLICATIONS

This application is related to and claims the benefit of U.S. Provisional Patent Application Serial Nos. 62/873,845 (“the '845 application”) and 62/873,846 (“the '846 application”), and U.S. Non-Provisional patent application Ser. No. 16/926,705 (“the '705 application”), each of which is titled “Scoliosis Brace,” and each of which is incorporated by reference herein in its entirety for all purposes. This application is a Continuation-In-Part of U.S. Non-Provisional patent application Ser. No. 16/926,705, which corresponds to U.S. Pat. No. 11,510,802 having an issue date of Nov. 29, 2022.

### BACKGROUND OF THE INVENTION

### Field of the Invention

The invention relates to methods and apparatus of orthopedic adjustment, correction, stabilization, and/or support, and in a particular embodiment, to a three-dimensional Tension-based Scoliosis Orthosis, commonly known as a Scoliosis Brace, such as might be used to stabilize, adjust, and/or correct a patient's spinal deformation, and to facilitate or restrict certain movements of human spine in the three different anatomical planes, i.e., coronal, sagittal and transverse planes. A preferred embodiment is directed to a tension-based scoliosis orthosis having a Y-shaped rigid back panel that generates continuous de-rotational forces in the transverse anatomical plane and translation forces in the coronal and sagittal planes. Proposed uses include orthopedic adjustment, correction, and/or stabilization of spinal deformations through a patient's daily, near-continuous, long-term wearing of the scoliosis brace.

### Description of Related Art

Scoliosis is among the most common disorders of the spine, affecting approximately 3 in every 100 people worldwide [1]. Scoliosis presents itself as a misalignment of the spinal column, which is generally defined as a curve that deviates from the central spinal axis by greater than 10 degrees. While often simply characterized as a lateral curvature in the frontal plane, a rotational deformation about the transverse plane is common. For this reason, the various forms of scoliosis are more accurately described as complex three-dimensional deformities with misalignment occurring in the frontal and sagittal planes, as well as rotation in the transverse plane.

Scoliosis is a complex three-dimensional deformity of the spine, and there are two main types of adult scoliosis, the idiopathic and degenerative. According with Scoliosis Research Society (SRS) the adult idiopathic scoliosis is a prolongation of the adolescent idiopathic scoliosis (AIS), and the adult “De novo” degenerative scoliosis is a type of scoliosis that begins during the adult life due to arthritis and degeneration of the musculoskeletal system [2].

Adults with scoliosis can have more and a greater variety of symptoms compared with adolescents and younger patients. The adults have more symptoms because of the degeneration of the soft and hard tissues of the musculoskeletal system. The most common symptoms of adult scoliosis patients are back pain, stiffness, numbness, cramping and shooting pain in the legs, that can lead to gradual loss of function.

Aebi et al. classified adult scoliosis into three major types [3], all leading to an asymmetric breakdown of spinal discs and facet joints. Adults scoliosis can stem from one or more pathologies. The Type 1-3 classification scheme provided by Aebi, et al. [3] categorizes each based on the pathology that is thought to have led to the scoliosis. The Type 1 and Type 3 are the most clinically relevant groups. The Type 1 adult scoliosis is the degenerative scoliosis and is first seen late in life (“de novo”) often after age 65 due to normal wear and tear on an aging spine. It is usually located in the thoracolumbar or lumbar spine and is often thought to express the mildest symptoms of the three cases [2][3].

The treatment options for adult scoliosis patients are the conservative (non-operative) treatment for patients that don't have disabling symptoms, and the operative treatment for patients that failed the non-operative therapies and for patients that have restricted functional activities and substantially have reduced overall quality of life [2].

Bracing is an important modality of the non-operative treatment of scoliosis, principally in treatment of Adolescent Idiopathic Scoliosis (AIS). The main goal of bracing therapy for AIS patients is to prevent the progression and/or correct the scoliosis curves during the bone growth period. A rigid thoraco-lumbo-sacral orthosis (TLSO) is a brace worn to minimize progression of AIS. There are various TLSO designs (e.g., Boston, Milwaukee, Wilmington) [4]. The Boston brace is one kind of the traditional rigid braces and is the most frequently prescribed scoliosis braces for adolescents with idiopathic scoliosis. One limitation about the Boston brace is that it weakens the muscles and stiffens the spine. There are studies that found the traditional TLSO does not correct the three-dimensional deformity even though it reduces the cobb angle [6].

One another brace that is used for the conservative treatment of AIS is the Rigo-Cheneau type brace. Historically, the Cheneau-type brace was designed to oppose the spinal torsion and correct scoliosis in three dimensions [7]. The Cheneau type brace was developed approximately twenty-five years ago, with the main goal to combine biomechanical forces in three different anatomical planes [5], including de-rotations of the scoliosis curve in the transverse plane. Typically, the Rigo-Cheneau type brace has an open pelvis design with anterior opening [5]. Lebel et al found that in-brace Apical Vertebral Rotation (AVR) of scoliosis curve was significantly reduced by the Cheneau brace when compared to the TLSO brace” [8].

The Rigo-Cheneau Brace has proven more effective in three-dimensional correction of spinal curvature in cases of idiopathic scoliosis when compared to other contemporary methods (and its technical principles will therefore be taken as a standard in our analysis). Each case of scoliosis has its own unique curvature profile and the brace design is different for each case. There are three biomechanical principles that are implemented in order to move the trunk into the best-balanced position. These principles are three-point system in the frontal plane, de-rotation in the transverse plane, and physiological alignment in the sagittal plane. The general technique is to achieve morphological 3D correction by using a combination of forces applied to the trunk surface through specifically designed pads, facilitated by expansion spaces. There is a corrective reaction of the body in response to these forces, resulting in more improved posture and spinal alignment [7].

Research shows that braces that provide three-dimensional corrective forces produce better outcomes to prevent the progression or correct scoliosis [5][7][8].

For adult patients, the main objectives of the bracing therapy are to provide relief of the symptoms and to promote a more balanced posture to improve the quality of life during daily living activities [2]. The existing scoliosis braces for adult patients available in the market don't provide an effective three-dimensional treatment of the scoliosis. This invention relates a three-dimensional tension-based scoliosis orthosis capable providing short term pain relief when in use and improvement of the spinal balance in scoliosis patients with different curve types. The disclosed bracing system is adjustable and personalized to provide the efficient three-dimensional correction to the specific curvature pattern of the patient.

### SUMMARY OF THE INVENTION

The invention is directed to systems, methods, and apparatus involving scoliosis bracing using a three-dimensional support system capable of providing pain relief and improvement of the spinal balance in scoliosis patients with different curve types. Embodiments of the disclosed bracing system include a tension-based scoliosis orthosis that may contain a rigid back panel. The rigid back panel is adapted to generate continuous de-rotational forces in the transverse anatomical plane and translation forces in the coronal and sagittal planes. The de-rotational thoracic, lumbar, and pelvic de-rotational supports/pads of the disclosed bracing system, provide facilitation or restriction of certain movements of human spine in the coronal, sagittal, and transverse planes. The scoliosis bracing system may be used in cases of, for instance, idiopathic scoliosis and degenerative scoliosis. Moreover, the invention is also adapted to be effective in treating postural abnormalities and unbalances of the spine, wherein the ability to maintain more balanced and improved posture of the trunk, pelvis, and shoulder girdle are desired.

This tension-based scoliosis system is adjustable and personalized to provide an efficient three-dimensional support to the specific curvature pattern of the patient. The adjustability of supportive/compression/pressure and tension forces are achieved by incorporating the design of the rigid back panel, attaching pads, and strapping system. This bracing system generates three points of pressure able to apply appropriate forces in the transversal plane, coronal plane, and sagittal plane, wherever needed for a given case as determined by the physician. With the appropriate determination of the direction and magnitude of the pressure from the bracing system, the rotational aspect of the scoliosis can also be corrected. The hole system provides sufficient pressure to support and stabilize an imbalanced trunk, and also restrict or facilitate the ability of patients to move. The mechanical properties of the back panel and strap system have a high strength to provide the appropriate support/compression and sufficient tension.

This tension-based scoliosis system provides also proprioceptive feedback of the trunk muscles, which helps to develop muscular memory so that the body can actively correct posture when the system is not in use.

The disclosed tension-based scoliosis system is lightweight and portable for frequent or daily use, while easily custom fitted. The design of donning this bracing system is simple, allowing the patients to easily wear and take off the brace every day without assistance.

The materials of this support system are biocompatible and hypoallergenic due to the possibility of direct contact with user's skin. To ensure proper hygiene the brace can be washable. The long-time wearing of the brace should not stiffen the muscle and cause inflammation of the skin. The brace design has also a function to reduce the side effects of insufficient blood supply and limited breath, which exist in current products. In order to mitigate pressure points, the brace design doesn't have any sharp edges or corners.

Further aspects of the invention are set forth herein. The details of exemplary embodiment of the invention are set forth in the accompanying drawings and description below. Other features, objects, and advantages of the invention will be apparent from the description and drawings, and from the claims.

### BRIEF DESCRIPTION OF THE DRAWINGS

By reference to the appended drawings, which illustrate exemplary embodiments of this invention, the detailed description provided below explains in detail various features, advantages, and aspects of this invention. As such, features of this invention can be more clearly understood from the following detailed description, considered in conjunction with the following drawings, in which the same reference numerals denote the same elements throughout. The exemplary embodiments illustrated in the drawings are not necessary to scale and are not to be considered limiting of its scope, for the invention may admit to other equally effective embodiments.

FIG. 1 shows a plan view image of a front inner side of an exemplary embodiment of the invention.

FIG. 2 shows a plan view image of a back outer side the exemplary embodiment of FIG. 1.

FIG. 3A shows a plan view image of a rigid back panel of the exemplary embodiment of FIG. 1, and FIGS. 3B and 3C show perspective view images of adjustable Velcro hook and loop system/fasteners on an inner face of a back piece that will attach with a lumbar belt of FIG. 1.

FIGS. 4A-4D show images of adjustment straps adapted to be connected to de-rotational pads of the tension-based scoliosis orthosis embodiment of FIG. 2.

FIG. 5A depicts an illustration of a typical posterior view of an x-ray of a person with a left lumbar/thoracolumbar scoliosis and the relationship of scoliosis concavity and convexity with different structural blocks of the spine. FIG. 5B depicts an illustration of directions of the rotation of the thoracic block, lumbar block, and pelvis block of a person with a left lumbar/thoracolumbar scoliosis curve. FIG. 5C depicts a typical posterior view of a body of a person with a left lumbar/thoracolumbar scoliosis.

FIG. 6A depicts a typical posterior view of a body of a person with a left lumbar/thoracolumbar scoliosis and its relationship with the convexity and concavity of the scoliosis curve. FIG. 6B depicts a slice of the transverse anatomical plane of patient body at the apex of a left lumbar/thoracolumbar scoliosis. FIG. 6C depicts an illustration of appropriate application of the de-rotational forces of the scoliosis bracing system to a person with a left lumbar/thoracolumbar scoliosis.

FIGS. 7A-7B depict de-rotation forces applied in a wearer body with left lumbar/thoracolumbar scoliosis. Each de-rotational force has two components, the component of the coronal plane and the component of the sagittal plane.

FIGS. 8A-8B show components of the de-rotational forces that the back panel of FIG. 3A is adapted to form in the coronal plane using a three-point pressure/force system.

FIGS. 9A-9B depict illustrations of components of the de-rotational forces that are to be applied in the sagittal plane.

FIGS. 10A-10C depict illustrations and an image of a preferred adjustment of the direction and magnitude of the de-rotational forces by using additional pads.

FIG. 11A depicts a typical posterior view of a body of a person with a left lumbar/thoracolumbar scoliosis and its relationship with the three-point pressure in the coronal plane. FIGS. 11B-11C show images of a setup of the disclosed tension-based scoliosis system for a person with a left lumbar/thoracolumbar scoliosis curve.

FIG. 12 shows images of a preferred sequence of a wearer donning the brace, in which the bracing system was adapted for a left lumbar/thoracolumbar scoliosis curve.

FIG. 13 shows images of an adjustment of strap tension of the de-rotational forces of a thoracic pad.

FIG. 14 shows images of an adjustment of strap tension of the de-rotational forces of a pelvis pad.

FIGS. 15A-15D show a setup of the tension-based scoliosis system for a person with right lumbar/thoracolumbar scoliosis.

FIGS. 16A-16D show a setup of the tension-based scoliosis system for a person with a right thoracic scoliosis curve.

FIGS. 17A-17D show a setup of the tension-based scoliosis system for a person with a left thoracic scoliosis curve.

FIGS. 18A-18B show images of an adjustment of a lumbar belt in the back panel for a person with a lumbar/thoracolumbar scoliosis curve.

FIGS. 19A-19B show images of an adjustment of a lumbar belt in the back panel for a person with a long thoracic scoliosis curve.

FIG. 20 shows a plan view of an inner side of an exemplary embodiment of a scoliosis brace, with images depicting exemplary potential directions of adjustment of the trunk section and branch sections and respective de-rotational pads.

FIG. 21 shows a plan view of an outer side of an exemplary embodiment of a scoliosis brace, having adjustable sections that are adapted to be adjusted with an exemplary section orientation adjustment mechanism and with two exemplary section length adjustment mechanisms, with images depicting exemplary potential directions of rotational orientation adjustment, horizontal adjustment, and length adjustment, of the trunk section and branch sections and respective de-rotational pads.

FIGS. 22A-22B show, respectively, a plan view of the outer side and a plan view of the inner side, of the exemplary embodiment of a scoliosis brace of FIG. 21.

FIGS. 23A-23D show, respectively, a plan view of the outer side of the exemplary embodiment of a scoliosis brace of FIG. 21; a plan view of a single, unattached branch section; a plan view of an unattached trunk section with an integrated central portion; and a plan view of an unattached trunk section with an integrated central portion, and having demarcations for potential length adjustments at a distal end of the trunk section.

FIGS. 24A-24C show, respectively, plan views of simplified block diagrams of exemplary embodiments aspects of a scoliosis brace, having a central portion with multiple attachment holes or locations adapted to function as a scaffold or attachment bus, to which would be attached the depicted-as-unattached trunk section, upper branch section, and lower branch section, each of which, along with the separate central portion, has a single row of attachment holes or locations, providing potential section vertical adjustability, but not otherwise providing potential section length adjustability or potential section orientation adjustability.

FIG. 25 shows a plan view of simplified block diagrams of exemplary embodiments of aspects of a scoliosis brace, having a central portion with multiple attachment holes or locations adapted to function as a scaffold or attachment bus, to which would be attached the depicted-as-unattached trunk section, upper branch section, and lower branch section, each of which, along with the separate central portion, has multiple rows of attachment holes or locations, providing potential section vertical adjustability, as well as providing potential section length or horizontal adjustability (by selecting one of several row depths, if only one row of holes is needed for secure attachment), and potential section orientation adjustability (when aligning holes at other-than right angles when orienting a section, if holes or attachment locations are configured to permit rotated attachments and still provide secure attachment).

FIGS. 26A-26C show plan views of simplified block diagrams of exemplary embodiments of aspects of unattached sections of a scoliosis brace, the sections depicted in differing sizes and having multiple rows of attachment holes or locations for attachment to a central portion, such as in FIG. 24 or FIG. 25, having attachment holes or locations adapted to function as a scaffold or attachment bus, to which would be attached the depicted-as-unattached trunk section, upper branch section, and lower branch section, each of which, along with the separate central portion, has multiple rows of attachment holes or locations, providing potential section vertical adjustability, as well as providing potential section length or horizontal adjustability (by selecting one of several row depths, if only one row of holes is needed for secure attachment), and potential section orientation adjustability (when aligning holes at other-than right angles when orienting a section, if holes or attachment locations are configured to permit rotated attachments and still provide secure attachment).

FIG. 27 shows a plan view of a simplified block diagram of an exemplary embodiment of aspects of sections of a scoliosis brace, each of the three sections comprising a durable elastic band, the three elastic bands connected at and/or attached to a central portion, and comprising the trunk section, upper branch section, and lower branch section, each of which providing potential section vertical adjustability, as well as providing potential section length or horizontal adjustability, and potential section orientation adjustability.

FIG. 28 shows a block diagram of an exemplary network environment of an exemplary embodiment of an electronic monitor for a scoliosis brace.

FIG. 29 shows a block diagram of circuitry of an exemplary embodiment of an electronic monitor for a scoliosis brace.

FIG. 30A shows an exemplary embodiment of an electronic monitor for a scoliosis brace, whereas FIG. 30B shows a block diagram of a workflow algorithm of launching an exemplary software application for the electronic monitor.

FIGS. 31A-31B show block diagrams of exemplary embodiments of workflow algorithms of a patient and of a tutor for an exemplary software application for the electronic monitor.

FIG. 32 shows a block diagram of an exemplary embodiment of a workflow algorithm of using an exemplary software application for the electronic monitor.

### LISTING OF DRAWING REFERENCES AND NUMERALS

Group 1—Back Panel
10—Y-shaped rigid/semi-rigid back panel
11—V1 De-rotational pad of the back panel
12—Y De-rotational pad of the back panel
13—V2 De-rotational pad of the back panel
14—“D” hole of the V1 De-rotational pad
15—“D” hole of the V1 De-rotational pad
16—Hole for the attachment of the lumbar belt—The lumbar belt will attach to the back panel through this hole.
17—The adjustable Velcro hook and loop system/fastener on the inner face of the back piece that will attach with the lumbar belt.
Group 2—Lumbar Belt
20—Semi-rigid lumbar belt
21—V arm of the lumbar belt
22—Y arm of the lumbar belt
23—Velcro Hook of the V arm of the lumbar belt
24—Cord system Y side of the lumbar belt
25—Cord system of the V side of the lumbar belt
26—Pull tab cord system of the V side of the lumbar belt that contains a Velcro hook that will attach to the Velcro loop fabric of the outer side of the belt.
27—Pull tab cord system of the Y side of the lumbar belt that contains a Velcro hook that will attach to the Velcro loop fabric of the outer side of the belt.
28—Cover of the cord system of the lumbar belt.
29—The outer side of the arms of the lumbar belt are made with a Velcro hook fabric.
210—The fabric permanently sewn in the lumbar belt that is going to attach to the back panel and attach to the cover of the back panel through.
211—The side of the strap 210 that is going to attach to the Velcro hook that is in the inner face of the back panel 17.
212—The Velcro hook of the strap 210 of the lumbar belt 20 that is going to attach to the Velcro loop 44 of the cover of the back panel
Group 3—Straps of de-rotational pads
31—V1 Strap of the de-rotation pad
32—V2 Strap of the de-rotational pad
33—Removable Velcro hook V1 strap
34—Removable Velcro hook V2 strap
35—Velcro hook permanently sewn on the distal end of V1 strap
36—Velcro hook permanently sewn on the distal end of V2 strap
37—Proximal end of the strap without the removable hook
38—Nylon fabric sewn in the distal edge of the strap
Group 4—Cover of back panel
40—Cover of the rigid/semi rigid back panel made of foam and polyester mesh
41—Elastic fabric to attach the cover made of foam and polyester mesh to the V1 de-rotational pad of the back panel.
42—Elastic fabric to attach the cover made of foam and polyester mesh to the V2 de-rotational pad of the back panel.
43—Elastic fabric to attach the cover made of foam and polyester mesh to the Y de-rotational pad of the back panel.
44—The posterior section of the back-panel cover is made from polyester fabric with Velcro loop and is in contact with inner face of the back panel.
50—Additional pad for attachment to a de-rotational support or support pad.

### DETAILED DESCRIPTION

Referring to the figures, exemplary embodiments of the invention and exemplary aspects of the invention are depicted to provide a better understanding of the description thereof presented herein.

### Exemplary Embodiments of a Scoliosis Brace

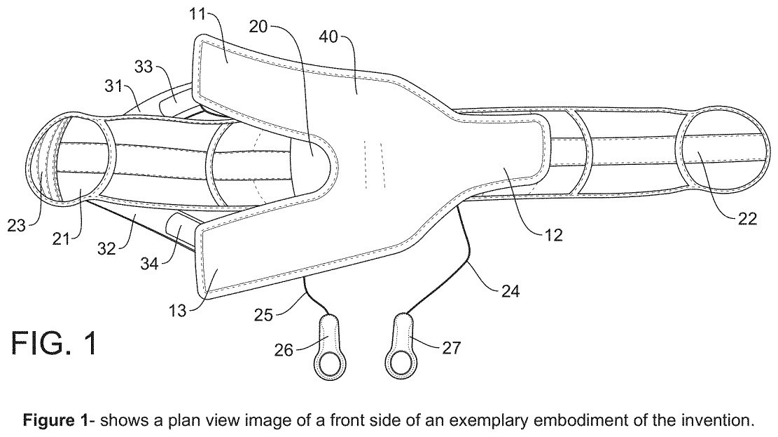

FIG. 1 shows a plan view of an inner side of an exemplary scoliosis brace that has been spread out, is not being worn by a wearer, and has not been adjusted to fit the wearer after the wearer dons the scoliosis brace. The inner side of the brace is the side that will face toward the wearer when worn and be adjacent the wearer when the wearer is wearing the brace.

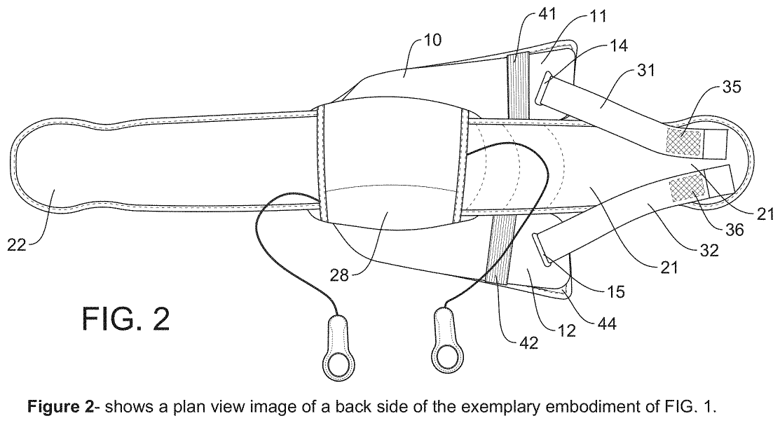

FIG. 2 shows a plan view of an outer side of the exemplary scoliosis brace of FIG. 1 that has been spread out, is not being worn by a wearer, and has not been adjusted to fit the wearer after the wearer dons the scoliosis brace. The outer side of the brace is the side that will face away from the wearer when worn and will not be adjacent the wearer when the wearer is wearing the brace.

As shown in FIG. 1 and FIG. 2, an exemplary of the tension-based scoliosis orthosis according to the invention may include, for example: a rigid/semi-rigid back panel 10; a lumbar belt 20 connected to the rigid back panel; a cover 40 of the rigid/semi-rigid back panel; and the V1 strap 31 and V2 strap 32 connected to the rigid/semi-rigid back panel. The V1 strap 31 and the V2 strap 32 and any other disclosed strap may be made, for instance, of durable nylon, polyester, rayon, cotton, leather, pliable resin, pliable rubber, woven fabric, etc.

FIGS. 1 and 2 show the V arm 21 and the Y arm 22 of the lumbar belt 20. FIG. 1 shows the sewn Velcro hook 23 in the V arm 21 of the lumbar belt. The fabric on the outer side of the lumbar belt is made from a fabric with Velcro loop, shown in FIG. 2.

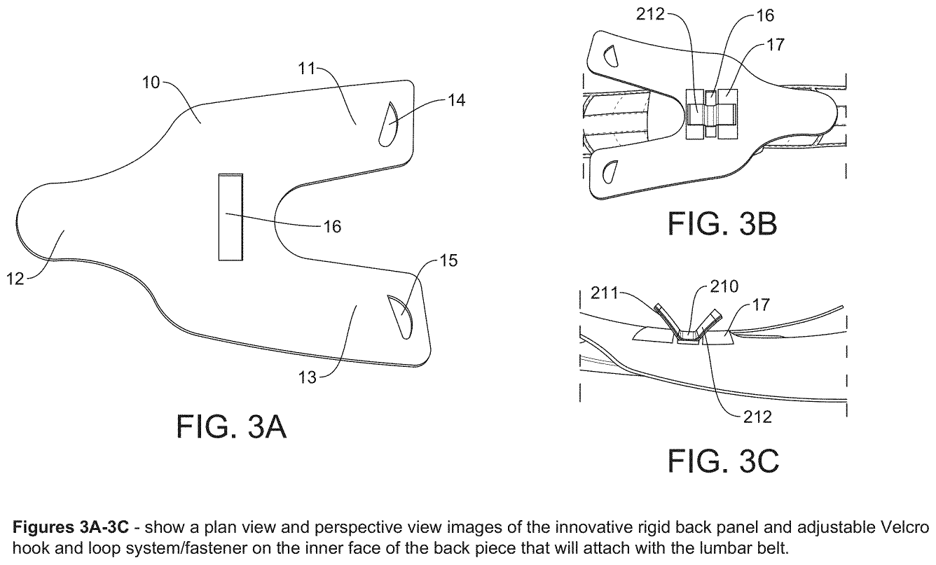

FIG. 3A shows the rigid/semi-rigid back panel 10. The back panel in one side has two de-rotational pads, the V1 de-rotational pad 11 and the V2 de-rotational pad 13 and in the other side has one de-rotational pad, the Y de-rotational pad 12. The back panel has a “D” hole 14 in the V1 de-rotational pad 11 and a “D” hole 15 in the V2 de-rotational pad. The V1 strap 31 and V2 strap 32 are going to attach to the de-rotational pads of the back panel through the “D” holes 14 and 15 respectively. FIG. 3A shows the hole 16 that was developed to create the adjustable Velcro hook and loop system/fastener.

FIGS. 3B and 3C show the adjustable Velcro hook and loop system/fastener 17 on the inner face of the rigid/semi-rigid back panel that will attach to the small durable nylon/polyester strap 210 permanently sewn in the lumbar belt 20. The rigid/semi-rigid back panel 10 and de-rotational straps 31 and 32 of this invention are the cores of the improved tension-based scoliosis orthosis and these components 10, 31 and 32 can be attached and adapted to other types of lumbar belts through the adjustable Velcro hook and loop system/fastener. FIGS. 3B and 3C show the side 211 of the strap 210 that is going to attach to the Velcro hook that is in the inner face of the back panel 17. FIG. 2 shows posterior section 44 of the cover 40 of the back panel. The posterior section 44 of the back-panel cover made from polyester fabric with Velcro loop and is in contact with inner face of the back panel. FIGS. 3B and 3C shows the Velcro hook 212 of the strap 210 of the lumbar belt 20 that is going to attach to the Velcro loop 44 of the cover of the back panel.

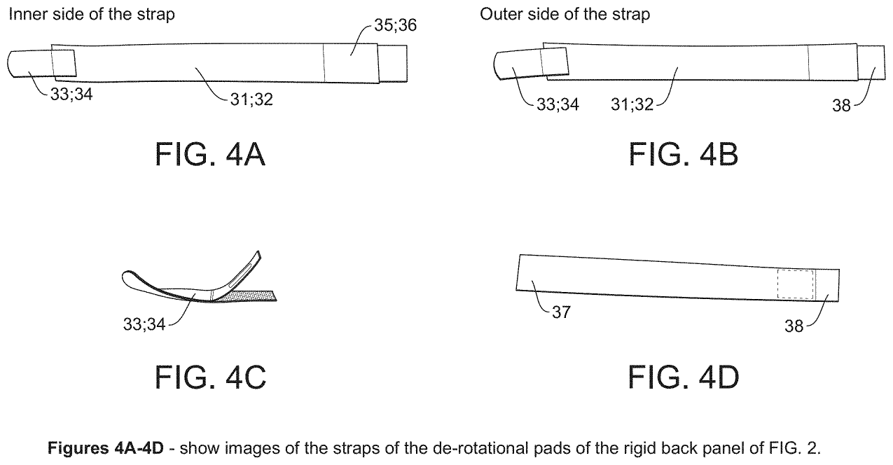

FIGS. 4A and 4B show the detailed view of V1 strap 31 and V2 strap 32 that are going to attach to the de-rotational pads of the back panel, 11 and 13 respectively and to the outer section of the V arm 21 of the lumbar belt 20. For custom fit and for an optimal adjustment of the tension of the straps V1 31 and V2 32, a health care professional can remove the Velcro hook piece 33 and/or 34 that is in the proximal end 37 of the strap shown in FIGS. 4C and 4D. The professional health care can customize/trim the size of the strap accordingly with the patient size, patient characteristics, scoliosis curve pattern and degree of deformity. After customization of the straps, the professional health care can setup, attach and adjust the straps in the de-rotational pads of the rigid/semi-rigid back panel 10 and lumbar belt 20. FIGS. 1 and 2 show the V1 strap 31 and V2 strap 32 attached to the rigid/semi-rigid back panel through the “D” holes 14 and 15. The removable Velcro hook 33 and 34 along the proximal end of the straps allows easy adjustment about the length. The Velcro hook pieces 35 and 36 permanently sewn on the distal end of V1 strap 31 and on the distal end of V2 strap 32 are going to be fixed in the Velcro loop of the outer side of V arm 21 of the lumbar belt 20, shown in FIG. 2.

To understand better the application of the disclosed invention is described an application of this improved tension-based scoliosis system to a body of a wearer with a left lumbar/thoracolumbar scoliosis.

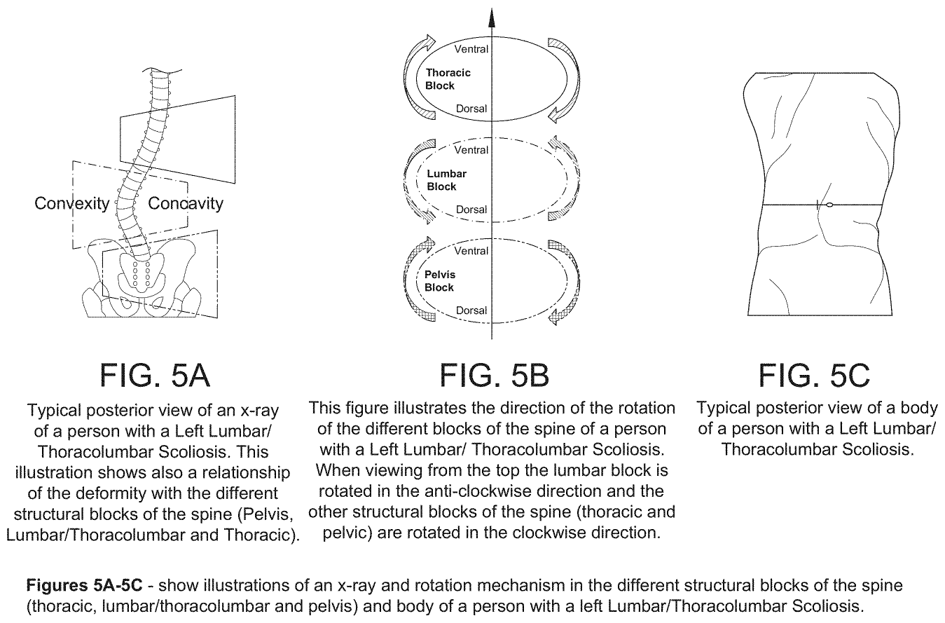

FIG. 5A shows a typical posterior view of an x-ray of a person with a left lumbar/thoracolumbar scoliosis and its relationship with the different structural blocks of the spine, pelvis, lumbar/thoracolumbar and thoracic. FIG. 5B illustrates a direction of the rotation of the structural blocks of the spine of a person with a left lumbar/thoracolumbar scoliosis. For this specific scoliosis curve pattern of a left lumbar/thoracolumbar curve, when viewing the transverse plane from the top the lumbar/thoracolumbar block is rotated in the anti-clockwise direction and the thoracic and pelvis blocks are rotated in the clockwise direction.

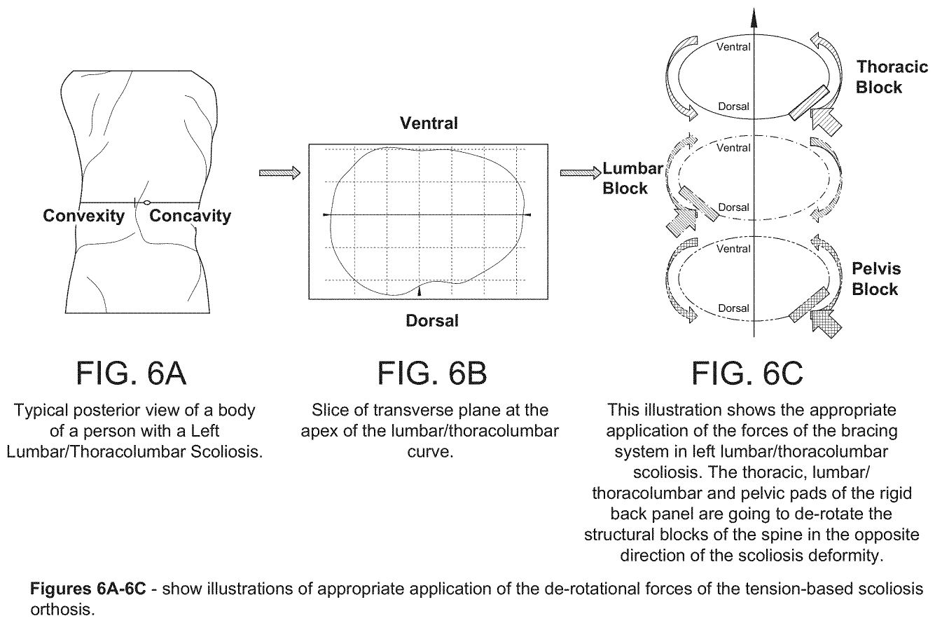

FIG. 6A shows typical posterior view of a deformed body of a person with a left lumbar/thoracolumbar scoliosis and its relationship with the convexity and concavity of the scoliosis curve. FIG. 6B shows an illustration of a slice of the transverse plane of the body of the patient at the apex of a left lumbar/thoracolumbar curve. FIG. 6C illustrates the appropriate application of the supportive forces of the bracing system in a body of a person with a left lumbar/thoracolumbar scoliosis. The thoracic, lumbar and pelvis pads are going to de-rotate the structural blocks of the spine in the opposite direction of the scoliosis deformity.

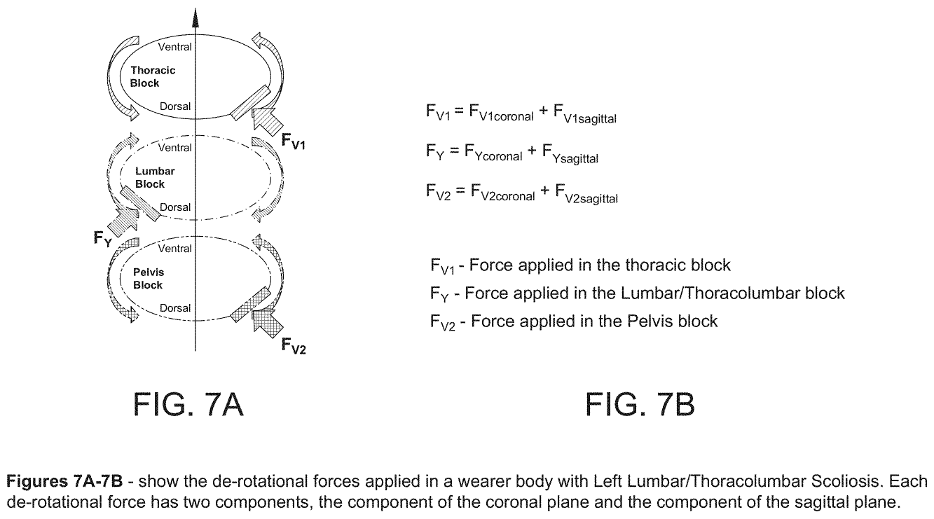

FIGS. 7A-7B illustrate the de-rotational forces that the disclosed tension-based scoliosis tension system is going to apply in a body of a wearer with a left lumbar/thoracolumbar scoliosis. The de-rotational forces are going to de-rotate the spine in the transverse plane and subsequent align the spinal segments in the frontal, transverse and sagittal planes.

Each de-rotational force has two components, the component of the coronal plane and the component of the sagittal plane. The V1 de-rotational pad 11 is going to apply the force Fv1 in the thoracic block, the Y de-rotational pad 12 is going to apply the force FY in the lumbar/thoracolumbar block and the V2 de-rotational pad 13 is going to apply the force FV2 in the Pelvis block.

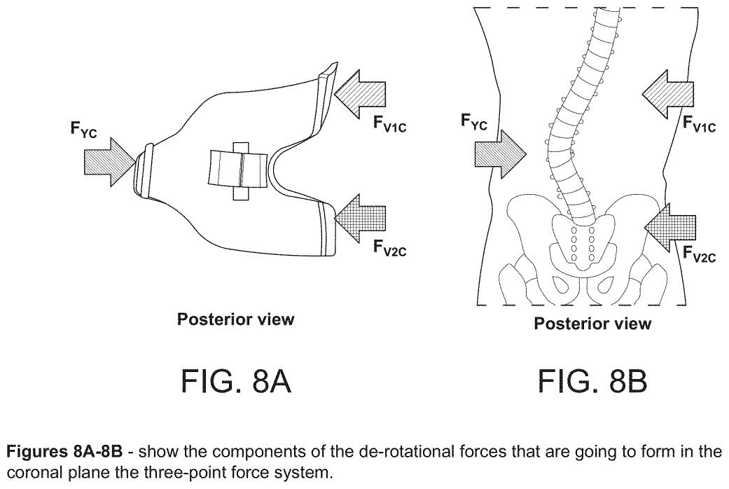

FIGS. 8A-8B show the components of the de-rotational forces that are going to form the three-point pressure system in the coronal plane. In traditional scoliosis bracing a three-point force system is formed by a corrective force and two counterforces applied proximally and distally as shown in FIG. 8B. The Y de-rotational pad 12 is going to apply a corrective/supportive force FYC at the apex of the convexity of the scoliosis curve. The V1 de-rotational pad 11 going to apply a counterforce Fv1C in the proximal end of the concave side of the of the scoliosis curve. The V2 de-rotational pad 13 is going to apply a counterforce Fv2C in the distal end of the concave side of the of the scoliosis curve.

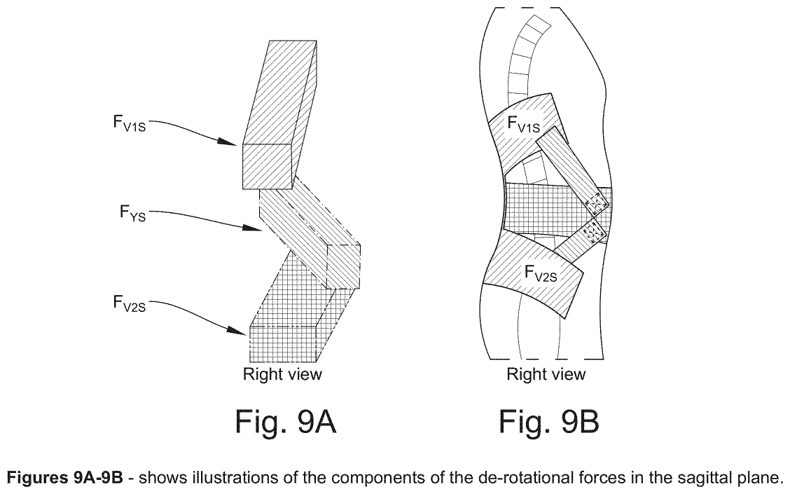

FIGS. 9A-9B show the components of the de-rotational forces in the sagittal plane. The force FV1S of the V1 de-rotation pad 11 is going to de-rotate the thoracic block in the anti-clockwise direction. The force FYS of the Y de-rotation pad 12 is going to de-rotate the lumbar block in the clockwise direction. The force FV2S of the V2 de-rotation pad 13 is going to de-rotate the pelvis block in the anti-clockwise direction.

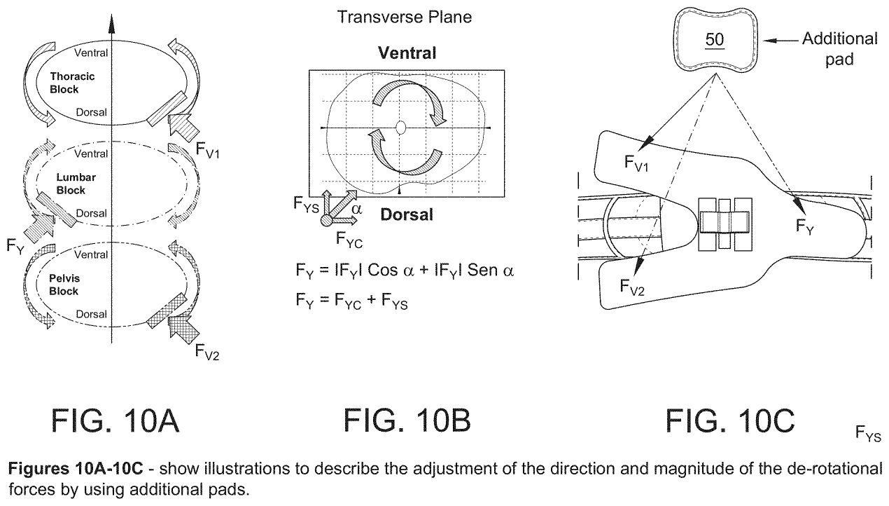

FIGS. 10A-10C show illustrations of how to adjust the direction and magnitude of the de-rotational forces by using additional pads. FIG. 10C shows the additional pad 50 that can be added and placed in the inner face of the rigid/semi-rigid back panel 10 and under the cover 40. The pad 50 may be placed more laterally or more posteriorly depending of the magnitude of the rotation or translation that the patient needs to achieve a more balanced position of the spine and improved posture.

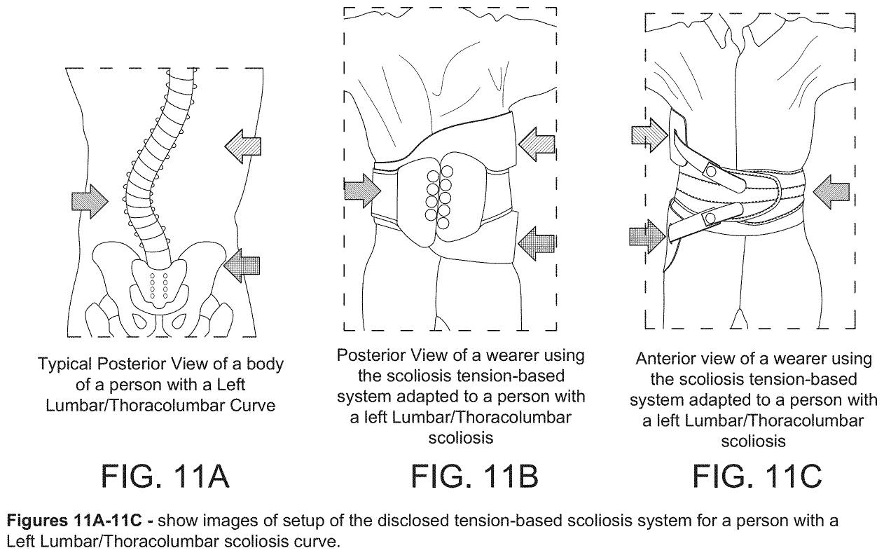

FIGS. 11A-11C show posterior and anterior view of the setup of the disclosed tension-based scoliosis system for a person with a left lumbar/thoracolumbar scoliosis. The geometric configuration and orientation of both straps 31 and 32 is highly adjustable. The bracing system is restricting the movement of side bending of the spine to the right side and facilitating the movement of side bending to the left. The tension-based scoliosis system is restricting the rotation of the thoracic block and pelvic block in the clockwise direction and facilitating the movement of rotation in the anti-clockwise direction. The tension-based scoliosis system is restricting the rotation of the lumbar/thoracolumbar block in the anti-clockwise direction and facilitating the movement of rotation in the clockwise direction.

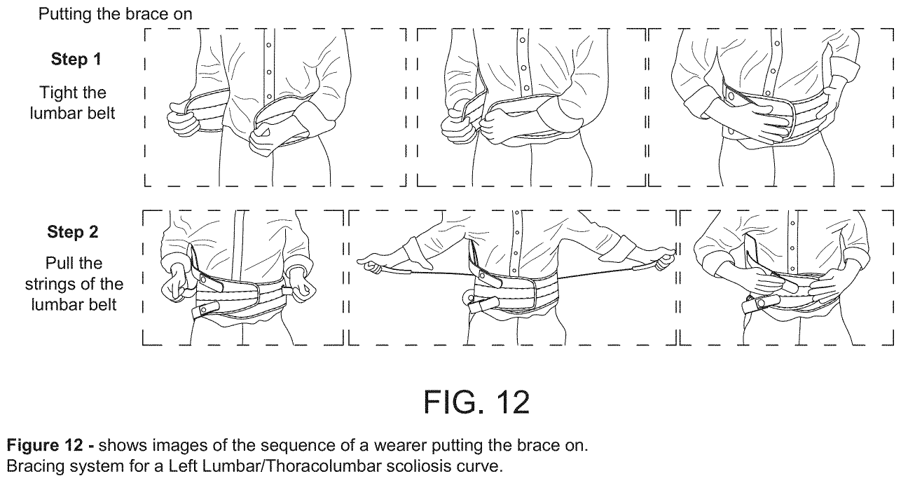

FIG. 12 shows the steps of a wearer putting the brace on. On step 1 the wearer holds the distal end of the V arm 21 and the distal end of the Y arm 22 of the lumbar belt 20 wrapping around the rigid/semi-rigid back panel 10 to provide consistent and strong support to the back, keeping the rigid/semi-rigid back panel 10 flush to the body of the wearer. There is a pulling system on the outer surface of the belt 20. The two pulling tabs 26 and 27 of are two major components of the pulling system, which are used to adjust the tightness of the belt. The belt 20 gets tighter as the tabs are being pulled. The pelvic belt 20 applies compression to torso at lumbar level to achieve lumbar alignment with the use of the sagittal profile of back panel 10. The compressive belt 20 transmits force to an Y de-rotational pad 13 of the rigid/semi-rigid back panel 10, de-rotating the lumbar block in the transverse plane in the clock-wise direction and creating a lateral force FYC in the coronal plane toward the medial axis, shown in FIGS. 8A-8B.

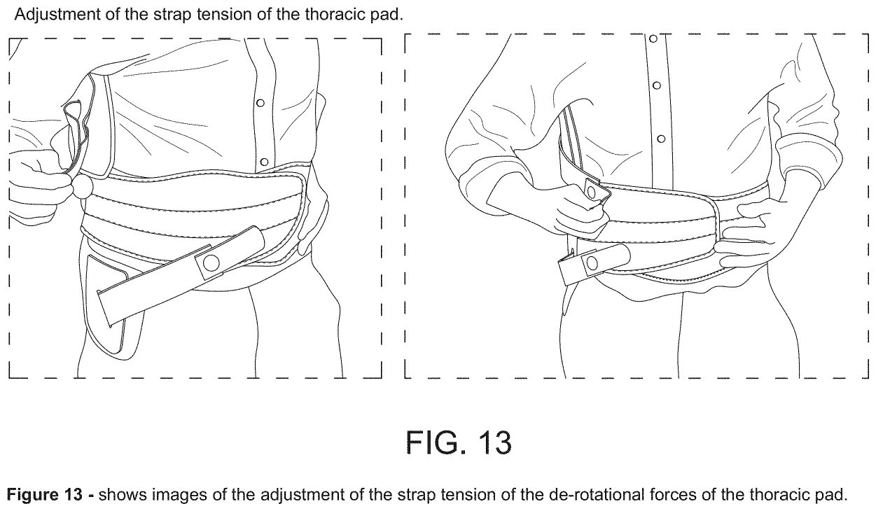

FIG. 13 shows the adjustment of the tension of the V1 strap 31 of the V1 de-rotational pad 11. Tightness of the V1 strap 31 can be adjusted by attaching the Velcro hook 35 on the different Velcro loop sections of the V arm 21 of the pelvic belt that are on the same level but different distance relative to the front center line. The V1 strap 31 that is attached to the V1 de-rotational pad 11 of the rigid/semi-rigid back panel 10, which impart force onto the right thoracic section of the body is de-rotating the thoracic block in the transverse plane in the anti-clockwise direction and creating a lateral force FV1C in the coronal plane toward the medial axis, as shown in FIGS. 8A-8B. As shown in FIGS. 10A-10C a pad may be placed in the inner face of the V1 de-rotational pad 11 to adjust the direction and/or increase the magnitude of the de-rotational force F V1.

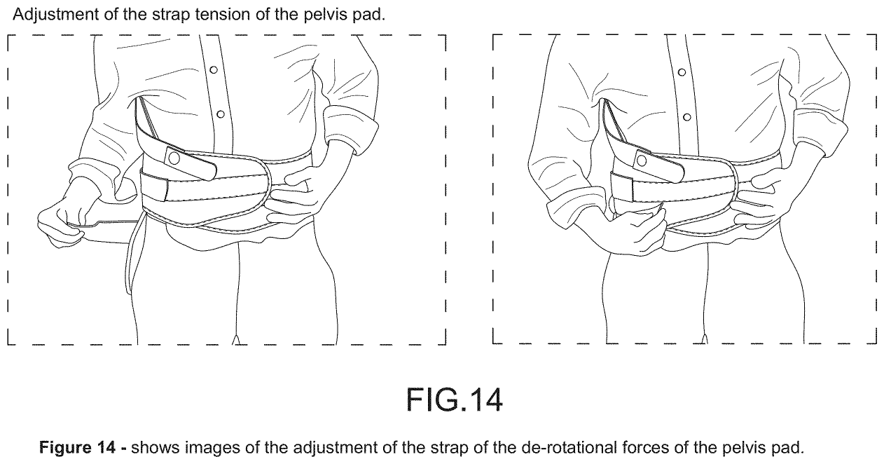

FIG. 14 shows the adjustment of the tension of the V2 strap 32 of the V2 de-rotational pad 13. Tightness of the V2 strap 32 can be adjusted by attaching the Velcro hook 36 on the different Velcro loop sections of the V arm 21 of the pelvic belt that are on the same level but different distance relative to the front center line. The V2 strap 32 that is attached to the V2 de-rotational pad 13 of the rigid/semi-rigid back panel 10, which impart force onto the right pelvis section of the body is de-rotating the pelvis block in the transverse plane in the anti-clockwise direction and creating a lateral force FV2C in the coronal plane toward the medial axis, as shown in FIGS. 8A-8B. As shown in FIGS. 10A-10C a pad may be placed in the inner face of the V2 de-rotational pad 13 to adjust the direction and/or increase the magnitude of the de-rotational force FV2. Each strap 31, 32 may be affixed and/or attached to a front surface of a ventral area of the lumber belt adjacent the wearer's torso when the lumber belt is donned/closed and worn.

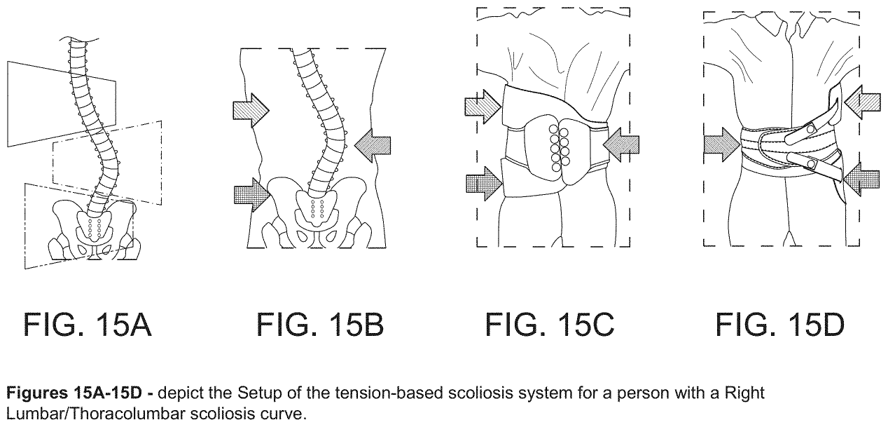

FIGS. 15A-15D show the setup of the disclosed tension-based scoliosis system for a person with a right lumbar/thoracolumbar scoliosis.

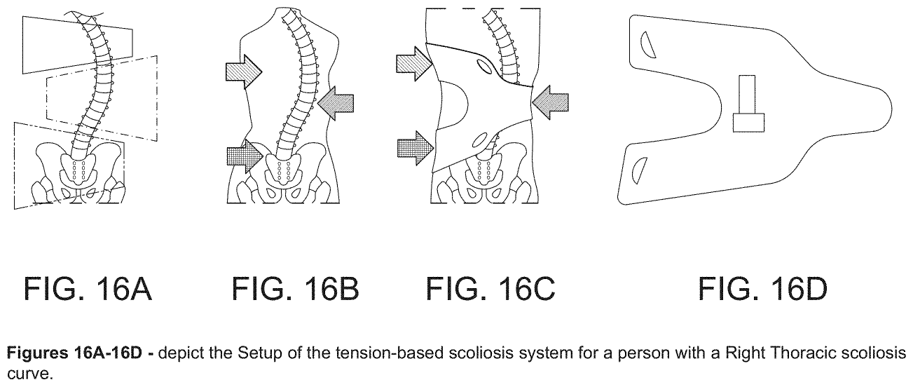

FIGS. 16A-16D show the Setup of the tension-based scoliosis system for a person with a Right Thoracic scoliosis curve.

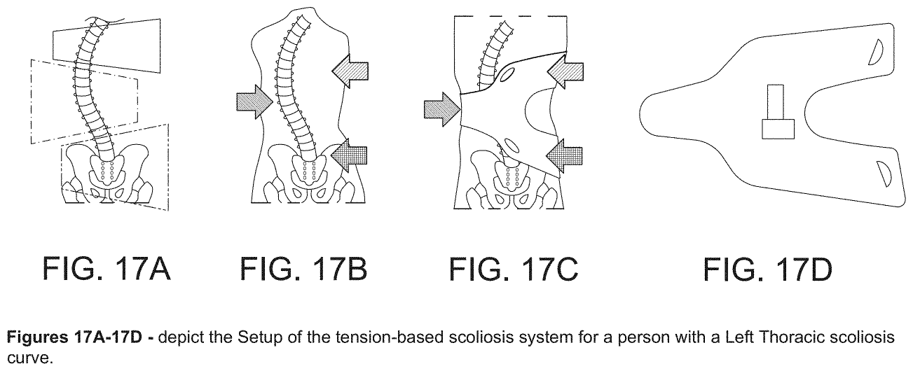

FIGS. 17A-17D show the Setup of the tension-based scoliosis system for a person with a Left Thoracic scoliosis curve.

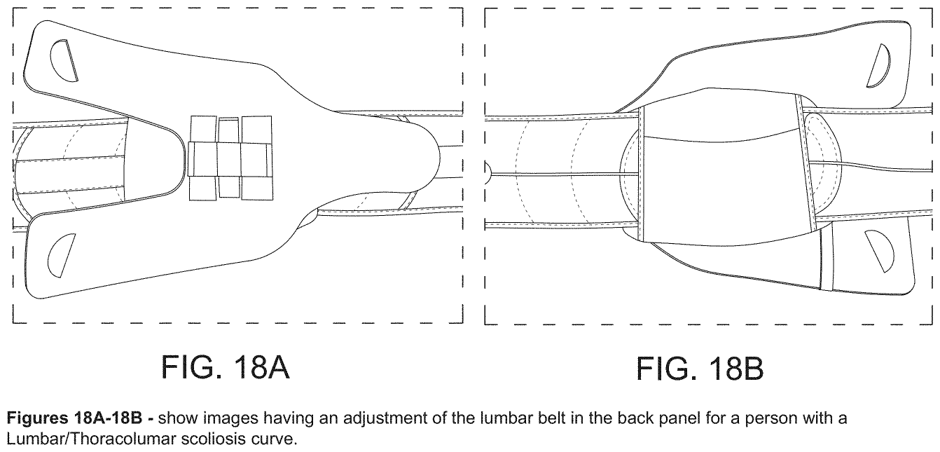

FIGS. 18A-18B show the adjustment of the lumbar belt 20 on the back panel 10 for a person with a Lumbar/Thoracolumbar scoliosis curve. The adjustment of the lumbar belt on the panel is made through the adjustable Velcro hook and loop system/fastener of the inner face of the back panel, as shown in FIGS. 3B and 3C. Typically for these curve types the apex of the convexity of the scoliosis curve is located at the level of the lumbar/thoracolumbar block. The belt has to fixed in the center of the hole 16 of the back panel, so the Y de-rotational pad 12 will match with the apex of the scoliosis curve.

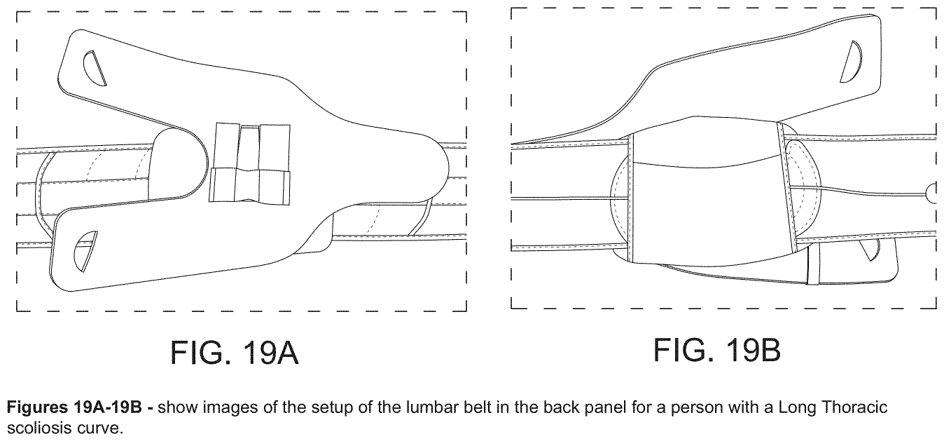

FIGS. 19A-19B show the adjustment of the lumbar belt 20 on of back panel 10 for a person with a long thoracic scoliosis curve. The adjustment of the lumbar belt on the panel is made through the adjustable Velcro hook and loop system/fastener of the inner face of the back panel, as shown in FIGS. 3B and 3C. Typically for these curve types the apex of the convexity of the scoliosis curve is located at the level of the thoracic block and we have to adjust the three-point pressure of the bracing system in higher level of the spine. The belt has to fixed in the lower section of the hole 16 of the back panel, so the Y de-rotational pad 12 will match with the apex of the scoliosis curve.

Adjustability of the Back Panel

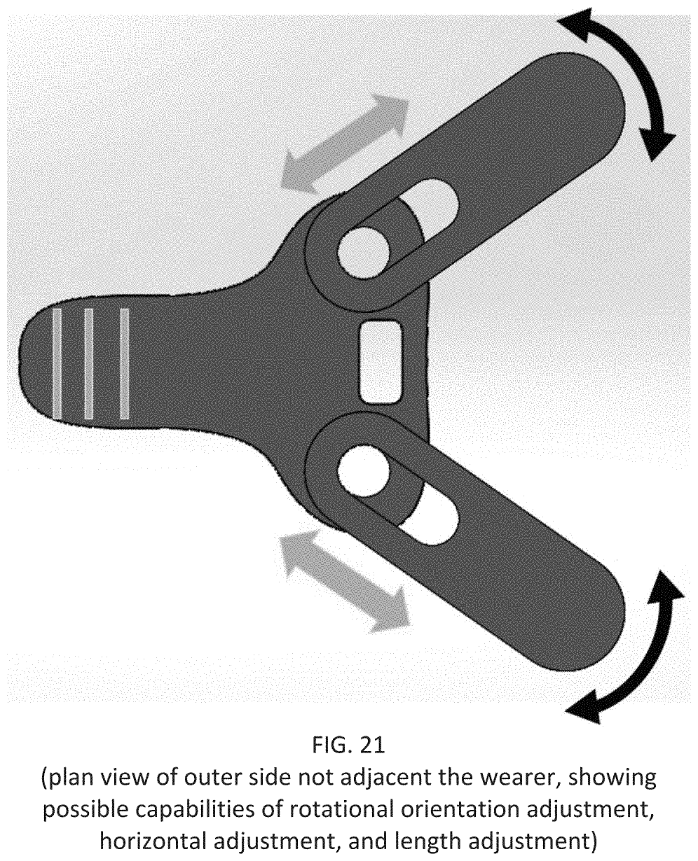

In some embodiments, the Y shape of the back panel 10 comprises a single integral piece of material, such as durable thermoplastic, as depicted in FIGS. 1-3C, 8A, 10C, 16D, 17D, 18A, and 19A. In embodiments having a single-substrate back panel 10, some embodiments likewise may have brace de-rotational pads combining into a single piece the de-rotational pads of the Y shape, combining and integrating the V1 de-rotational pad 11, the Y de-rotational pad 12, and the V2 de-rotational pad 13 as depicted, for instance, in FIG. 3A and FIG. 8A. Similarly, in embodiments having a single-substrate back panel 10, some embodiments likewise may have a brace cover 40 combining into a single assembly, piece, or lining the cover 40 and elastic fabric 41, 42, 43 on the Y shape, such as combining the elastic fabric 41 on the V1 de-rotational pad 11, the elastic fabric 42 on the Y de-rotational pad 12, and the elastic fabric 43 on the V2 de-rotational pad 13 as depicted in FIGS. 1-2. However, a single-substrate back panel nonetheless may have a multiple-piece cover, or multiple cover pieces comprising a cover assembly, as in FIG. 21 et seq.

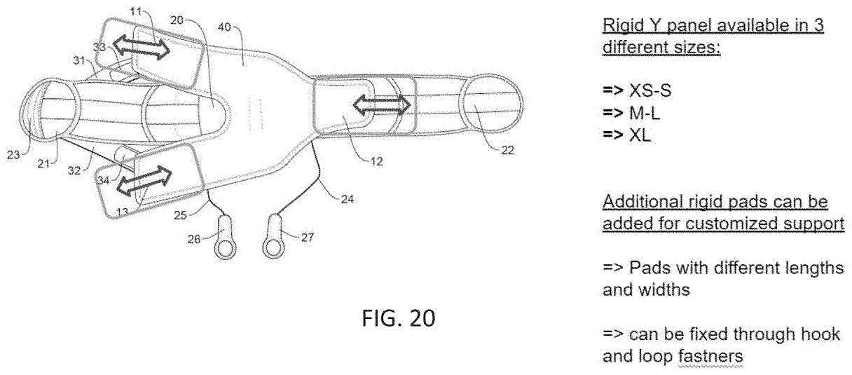

Embodiments having a back panel 10 comprising a single substrate, and the de-rotational pads on the single substrate, covered by a single cover or lining, may be beneficial for ease of use and limited risks of misunderstandings of methods of use, due to the adjustabilities of the brace mainly comprising the adjustment of the lumbar belt 20 and adjustment of the straps 33, 34 fitting the distal ends of the back panel and de-rotational pads around the wearer, as depicted in FIGS. 12-14. Such embodiments, though, may require multiple commercial products, such as described and depicted in FIG. 20, to suit various body sizes, shapes, spine deformations, and scoliosis diagnoses, to the extent that such back panel and de-rotational pad are fixed sizes per commercial product embodiment.

FIG. 20 shows a plan view of an inner side of an exemplary embodiment of a scoliosis brace, with images depicting exemplary potential directions of adjustment of the trunk section and branch sections and respective de-rotational pads. As depicted in FIG. 20, a rigid Y shaped back panel may be made available commercially in various sizes, e.g., extra-small (“XS”), small (“S”), medium (“M”), large (“L”), extra-large (“XL”), and combinations and/or consolidations thereof. Moreover, as shown in FIG. 10C, additional rigid pads 50 can be added, removed, and/or repositioned for more-customized support. The additional pads may comprise, for example, a rigid core, for support, covered in soft fabric, for improved tolerability. The pads may be of different lengths, widths, and thicknesses to further customize the support. Customized pads may be attached and/or affixed using, for example, hook-and-loop fasteners, snaps, laces, or similarly attachment mechanisms.

In some other embodiments, the Y shape of the back panel comprises multiple integrated pieces or substrates of material, such as durable thermoplastic or semi-pliable metal, or combinations of materials, as depicted in FIG. 21 et seq. In embodiments having a multiple-substrate back panel, some embodiments likewise may have brace de-rotational pads combining in use, but separate in assembly as single pieces, the de-rotational pads of the Y shape, combining and assembling the V1 de-rotational pad, the Y de-rotational pad, and the V2 de-rotational pad as depicted in FIG. 21 et seq. Similarly, in embodiments having a multiple-substrate back panel, some embodiments likewise may have a brace cover combining into a single cover assembly and/or multiple covers assemblies, as separate pieces or linings, the cover and elastic fabric on the Y shape, such as separating the elastic fabric on the V1 de-rotational pad, the elastic fabric on the Y de-rotational pad, and the elastic fabric on the V2 de-rotational pad as depicted in FIG. 21 et seq. However, a multiple-substrate back panel nonetheless may have a single-piece cover 40, as in FIG. 1.

Embodiments having a back panel comprising multiple substrates, and the de-rotational pads on the multiple substrates, each covered by a separate cover or lining, may be beneficial for flexibility of use and variability of methods of use, due to the adjustabilities of the brace comprising not just the adjustment of the lumbar belt and adjustment of the straps fitting the distal ends of the back panel around the wearer, as depicted, for instance, in FIGS. 12-14, but also the adjustabilities of the sections of the back panel. Such embodiments may enable one commercial product to suit multiple various body sizes, shapes, spine deformations, and scoliosis diagnoses, to the extent that such back panel, de-rotational pads, and cover(s) may be adjustable and combinable in size and shape per commercial product embodiment. Inasmuch as the added adjustability of such embodiments of the scoliosis brace increase the permutations of possible adjustments, the increase in permutations of possible adjustments likely makes the brace more complex and complicated to properly adjust to an intended wearer. Such increased complexity in the adjustment process lends itself to having only trained clinicians adjust the brace for the intended wearer, and preferably making the adjustments in a controlled clinical office environment having appropriate tools and measurement capabilities to accurately determine and implement the appropriate adjustments for the intended wearer. Once professionally adjusted, however, the scoliosis brace should be simple and easy to don and wear for an average adult-age wearer.

In embodiments having the Y shape comprised of multiple integrated pieces or substrates, the pieces comprising the sections of the Y shape comprise a trunk section and two branch sections. The trunk section and the two branch sections are connected at and to a central portion. The central portion may connect to the lumbar belt adapted to be secured to and around a wearer's waist, back, and abdomen. When the Y shape is disposed horizontally on a lateral side of the Y shape, the two branch sections comprise an upper branch section and a lower branch section, possibly extending up and down, respectively, relative to the central portion and/or the trunk section. Extension and orientation of the trunk section, the upper branch section, and the lower branch section may depend also on a section connection of the section to the central portion, and a section orientation relative to one section and the central portion, as determined by the section connection. Connections between the sections and/or central portion may comprise adjustable attachment mechanisms, such as lockable gears, tightenable winches, threaded screws, threaded-screw knobs, threaded bolts, threaded nuts, pins, axles, rivets, slidable bars, lockable slots, lockable flanges, spring-loaded or spring-biased engagements or releases, laced cords, rubber strips, elastic bands, and/or combinations thereof and/or of similar devices and mechanisms known in the art.

FIG. 21 shows a plan view of an outer side of an exemplary embodiment of a scoliosis brace, having adjustable sections that are adapted to be adjusted with an exemplary section orientation adjustment mechanism and with two exemplary section length adjustment mechanisms, with images depicting exemplary potential directions of rotational orientation adjustment, horizontal adjustment, and length adjustment, of the trunk section and branch sections and respective de-rotational pads.

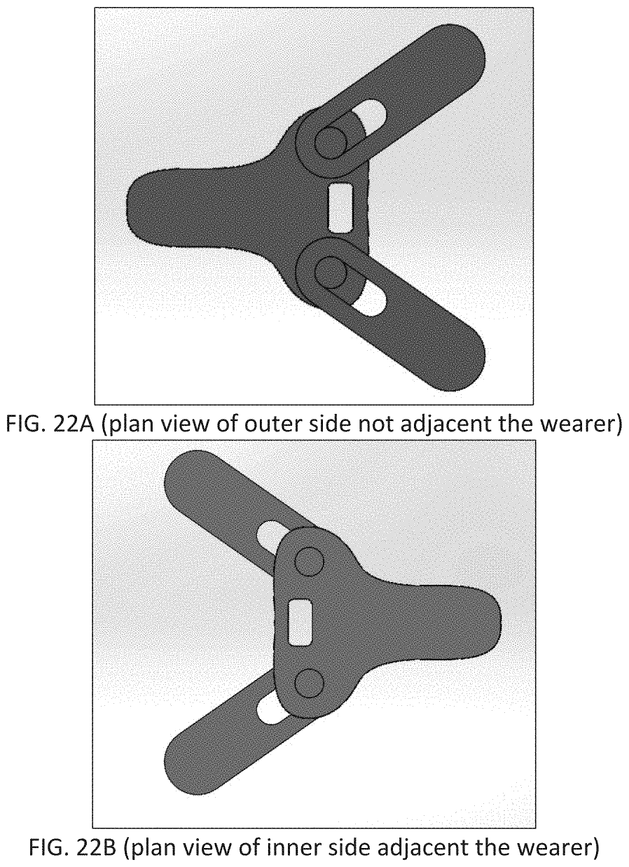

FIGS. 22A-22B show, respectively, a plan view of the outer side and a plan view of the inner side, of the exemplary embodiment of a scoliosis brace of FIG. 21.

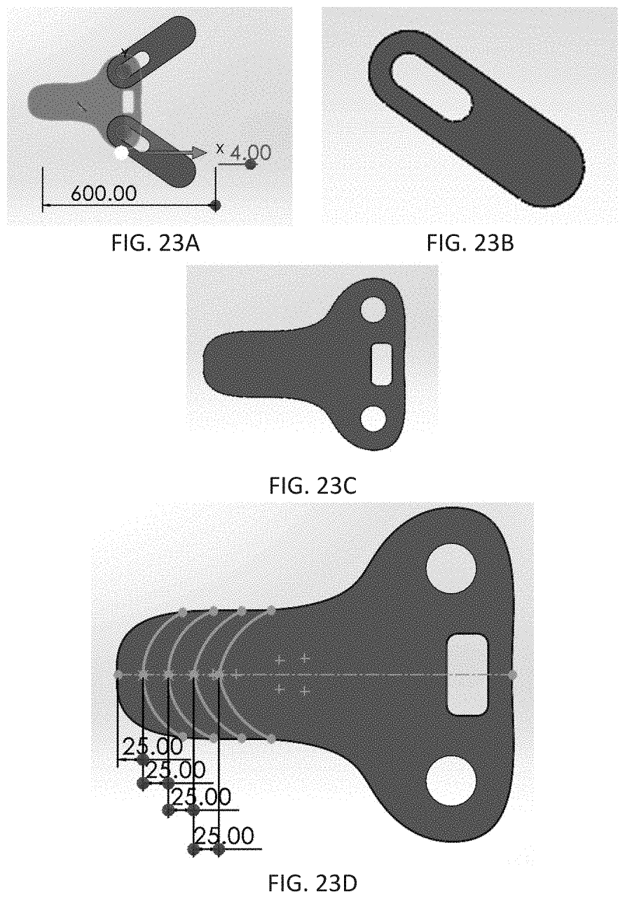

FIGS. 23A-23D show, respectively, a plan view of the outer side of the exemplary embodiment of a scoliosis brace of FIG. 21; a plan view of a single, unattached branch section; a plan view of an unattached trunk section with an integrated central portion; and a plan view of an unattached trunk section with an integrated central portion, and having demarcations for potential length adjustments at a distal end of the trunk section.

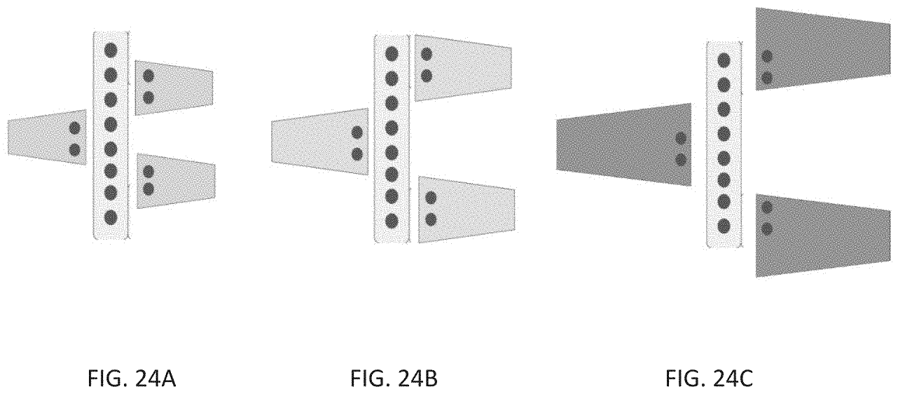

In some embodiments, the central portion is a part of the trunk section or one of the two branch sections, such as depicted, for instance, in FIGS. 21-23D. In some other embodiments, the central portion is a separate piece or structure disposed between the trunk section and the two branch sections, such as depicted, for instance, in FIG. 24A-26C. In either case, one or more sections may be adjustably connected to the central portion and adjustably positioned relative to the central portion and the other sections. For example, a separate central may comprise one hub, or multiple hubs, analogous to a hub of a hub-and-spoke wheel, with each of the sections comprising a spoke, also analogous to said hub-and-spoke wheel. Using an adjustable orientation adjustment mechanism connecting the central portion and one or more sections, an adjustable section orientation may be adjusted by rotating the section up or down, similar to movements of clock hands on a clock dial, such as depicted, for instance, in FIG. 21. In other embodiments, a separate central portion may comprise an attachment bus, hub, axle, or scaffold, such as having multiple attachment locations for different positions of attachment of the sections, adapting the Y shape back panel to be have adjustable sections that might be adjusted rotationally, as with clock hands, as well as vertically, such as increasing or decreasing a separation distance between the upper branch section and the lower branch section, and horizontally, such as increasing or decreasing a section length of the trunk section, the upper branch section, and/or the lower branch section.

Each section of the Y shape has a section length from a central portion of the Y shape of the back panel. The section length corresponds to a distance from the central portion of the back panel to a distal end of the section. The section length does not necessarily correspond to a length of a piece of material or substrate that comprises the section, because a proximate end of the section, adjacent to and/or near the central portion, may overlap with the central portion, reducing the section length of the section extending beyond the central portion. The proximate end of the section piece or substrate may overlap with the central portion for one or more reasons, such as to secure the section to the central portion, to adjust a section orientation of the section relative to the central portion or relative to the other sections, and/or to adjust the section length of the section extending beyond the central portion. As depicted, for instance, in FIG. 21, the two branch sections have section lengths that may be adjusted separately by sliding the section inward and outward with a hub at the central portion within a slot on the section.

In addition, the section length may be adjustable at the distal end of the section. As depicted, for instance, in FIG. 21, the trunk section has a section length that may be adjusted separately by removing, detaching, or severing a distal end of the section along one or more designated break-points on the section. Said break-points might comprise removable-pin hinges, perforation lines, or etch lines, or similar adjustment mechanisms, adapted to allow the de-rotational pad of the trunk section to remain durable during wearing to appropriately hold the wearer's body in the desired position without the distal end inadvertently breaking off on its own during use.

Adjustability of the back panel of the section length and/or section orientation may increase the flexibility of use of the brace and the variability of the methods of use of the brace among different wearers. Such a back panel with an adjustable Y shape therefore may be adjustable to suit a wearer and the wearer's personal scoliosis needs, and the adjustment may make the Y shape longer (e.g., longer trunk section and/or longer branch sections), shorter (e.g., shorter trunk section and/or shorter branch sections), wider (e.g., obtusely-angled/oriented branch sections), narrower (e.g., acutely-angled/oriented branch sections), non-symmetrical (e.g., differing section lengths and/or differing angles between the trunk section and each branch section), bent (e.g., a trunk section oriented up or down and bent relative to the central portion, and having differing angles between the trunk section and each branch section), offset (e.g., the central portion is offset relative to the wearer's spine and sides), and/or any combination thereof. As depicted clearly, for instance, in FIG. 8A, the back panel and section ends may be curved forward to conform to the curvature of the wearer's waist, back, and abdomen. In addition, the sections themselves may be curved upward and/or downward, such that a curved section is not straight from the proximate end to the distal end.

FIGS. 24A-24C show, respectively, plan views of simplified block diagrams of exemplary embodiments of aspects of a scoliosis brace, having a central portion with multiple attachment holes or locations adapted to function as a scaffold or attachment bus, to which would be attached the depicted-as-unattached trunk section, upper branch section, and lower branch section, each of which, along with the separate central portion, has a single row of attachment holes or locations, providing potential section vertical adjustability, but not otherwise providing potential section length or horizontal adjustability or potential section orientation adjustability.

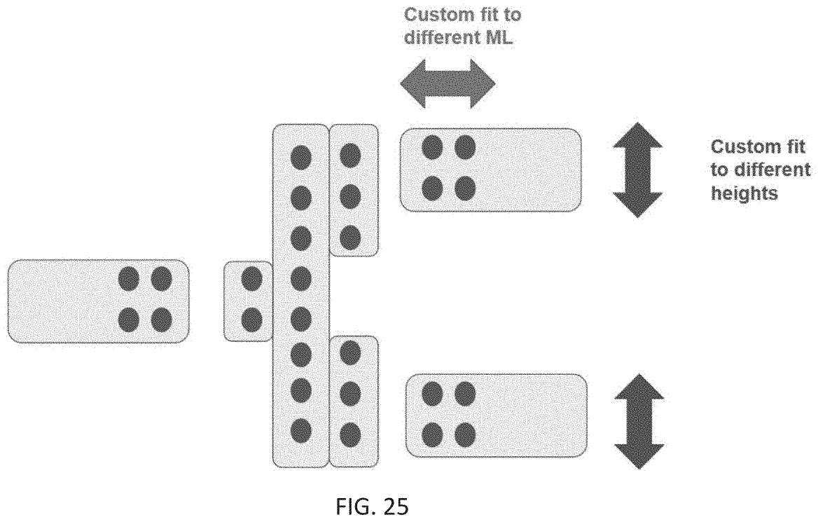

FIG. 25 shows a plan view of simplified block diagrams of exemplary embodiments of aspects of a scoliosis brace, having a central portion with multiple attachment holes or locations adapted to function as a scaffold or attachment bus, to which would be attached the depicted-as-unattached trunk section, upper branch section, and lower branch section, each of which, along with the separate central portion, has multiple rows of attachment holes or locations, providing potential section vertical adjustability, as well as providing potential section length or horizontal adjustability (by selecting one of several row depths, if only one row of holes is needed for secure attachment), and potential section orientation adjustability (when aligning holes at other-than right angles when orienting a section, if holes or attachment locations are configured to permit rotated attachments and still provide secure attachment).

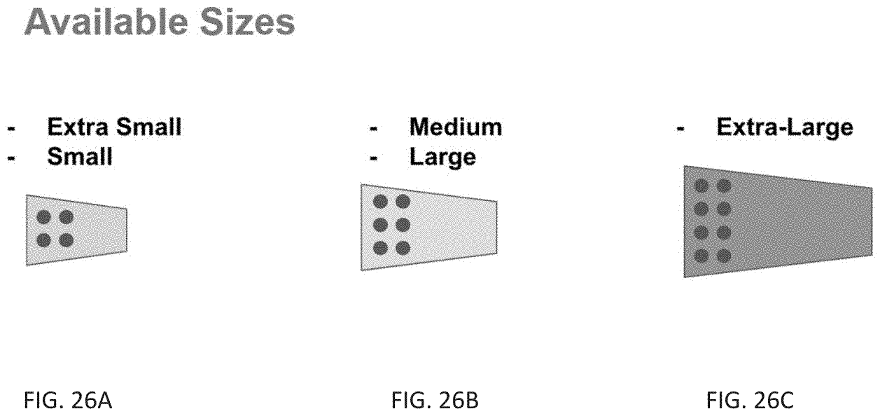

FIGS. 26A-26C show plan views of simplified block diagrams of exemplary embodiments of aspects of unattached sections of a scoliosis brace, the sections depicted in differing sizes and having multiple rows of attachment holes or locations for attachment to a central portion, such as in FIG. 24 or FIG. 25, having attachment holes or locations adapted to function as a scaffold or attachment bus, to which would be attached the depicted-as-unattached trunk section, upper branch section, and lower branch section, each of which, along with the separate central portion, has multiple rows of attachment holes or locations, providing potential section vertical adjustability, as well as providing potential section length or horizontal adjustability (by selecting one of several row depths, if only one row of holes is needed for secure attachment), and potential section orientation adjustability (when aligning holes at other-than right angles when orienting a section, if holes or attachment locations are configured to permit rotated attachments and still provide secure attachment).

Separately, in some embodiments of a scoliosis brace, durable elastic bands, possibly similar to elastic bands used in exercise training, may be used with and/or part of a back panel, with the elastic bands either positioned adjacent a wearer's back and applying inward-and-backward tension to the de-rotational pads, or positioned adjacent a wearer's front ventral abdomen and applying inward-and-forward tension to the de-rotational pads. If an elastic band is used as a section of the back panel, e.g., the trunk section, upper branch section, or lower branch section, the elastic band may be attached to the central portion and to the corresponding de-rotational pad. Due to the flexibility of the elastic bands, the elastic bands may provide adjustability of the section length, which may be adjustable at the distal end of the section, as well as adjustability of horizontal, vertical, and rotational orientation of the section and corresponding de-rotational pad (keeping in mind that the straps 31, 32 will hold the elastic bands and de-rotational pads in place by wrapping around the wearer wearing the brace and attaching to the lumbar belt at a front surface.

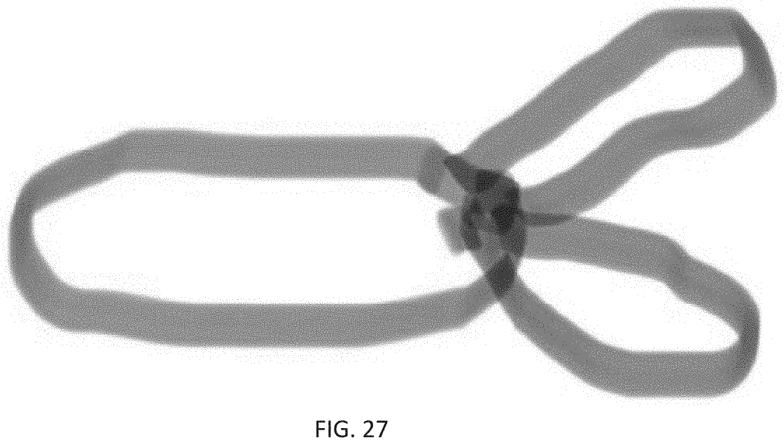

FIG. 27 shows a plan view of a simplified block diagram of an exemplary embodiment of aspects of sections of a scoliosis brace, each of the three sections comprising a durable elastic band, the three elastic bands connected at and/or attached to a central portion, and comprising the trunk section, upper branch section, and lower branch section, each of which providing potential section vertical adjustability, as well as providing potential section length or horizontal adjustability, and potential section orientation adjustability. Separately, the assembly of three elastic bands connected and/or attached at a central portion may be used on a wearer's front ventral abdomen to provide additional support to, by attaching or connecting to, the de-rotational pads, supplementing the support from the straps 31, 32, and pulling forward and inward the de-rotational pads that are positioned using the placement of the straps, the positioning of the lumbar belt, and the configuration of the back panel.

Monitoring of Tension and Use of the Back Panel

In some embodiments, the scoliosis brace includes an electronic monitor attached to the lumbar belt and/or one or more straps of the de-rotational pads. The electronic monitor may be characterized as a “smart” monitor in the sense that the monitor may include computer processing circuitry and preferably wireless connectivity hardware and software (e.g., WiFi, Near-Field Communication (“NFC”), and/or Bluetooth transceiver input/output modules), and possibly wired connectivity hardware and software (e.g., a USB port for data transfer and/or charging an internal battery as an integrated power source). The electronic monitor is adapted to monitor relative tension in and/or between attached straps and other attachments, in which the monitor may include an electronic tension measurement device, such as a piezoelectric pressure/tension sensor. Other electronic sensors may include accelerometers to detect motion, position, and orientation of a wearer when wearing the monitor and an attached scoliosis brace.

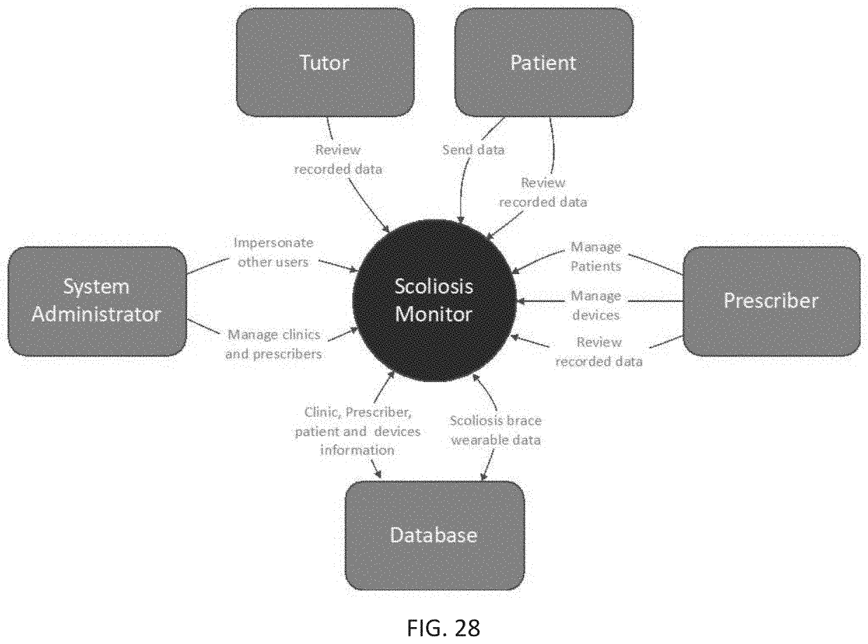

FIG. 28 shows a block diagram of an exemplary network environment of an exemplary embodiment of an electronic monitor for a scoliosis brace.

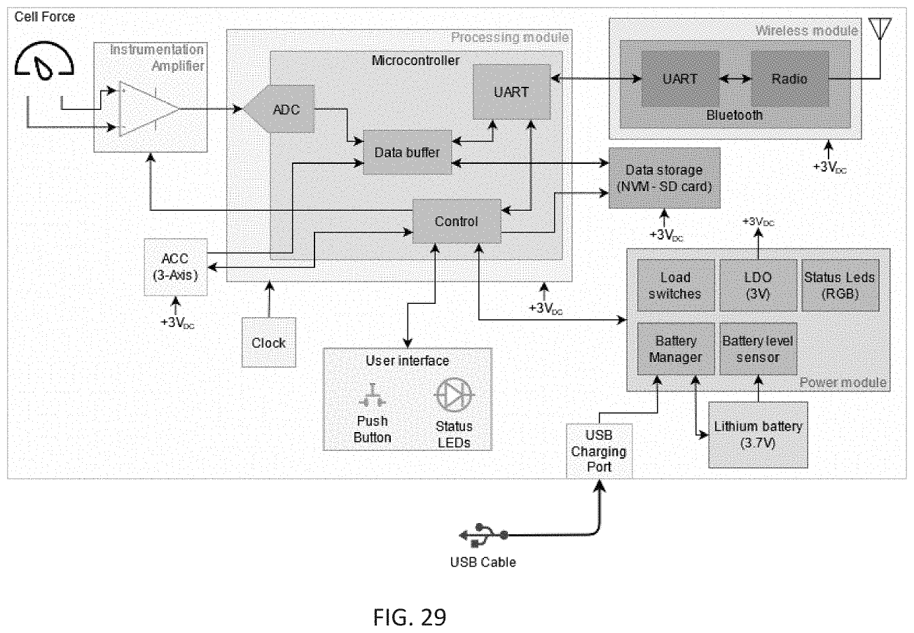

FIG. 29 shows a block diagram of circuitry of an exemplary embodiment of an electronic monitor for a scoliosis brace.

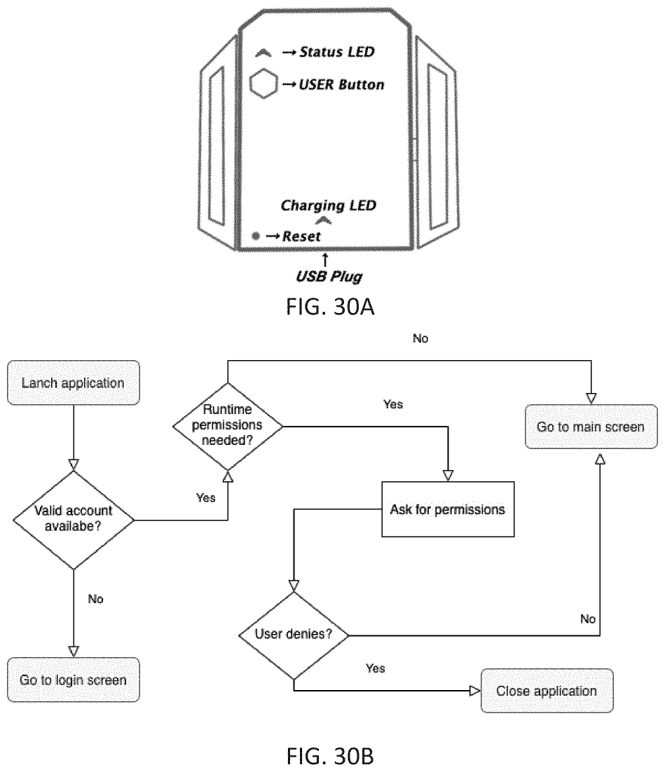

FIG. 30A shows an exemplary embodiment of an electronic monitor for a scoliosis brace, whereas FIG. 30B shows a block diagram of a workflow algorithm of launching an exemplary software application for the electronic monitor.

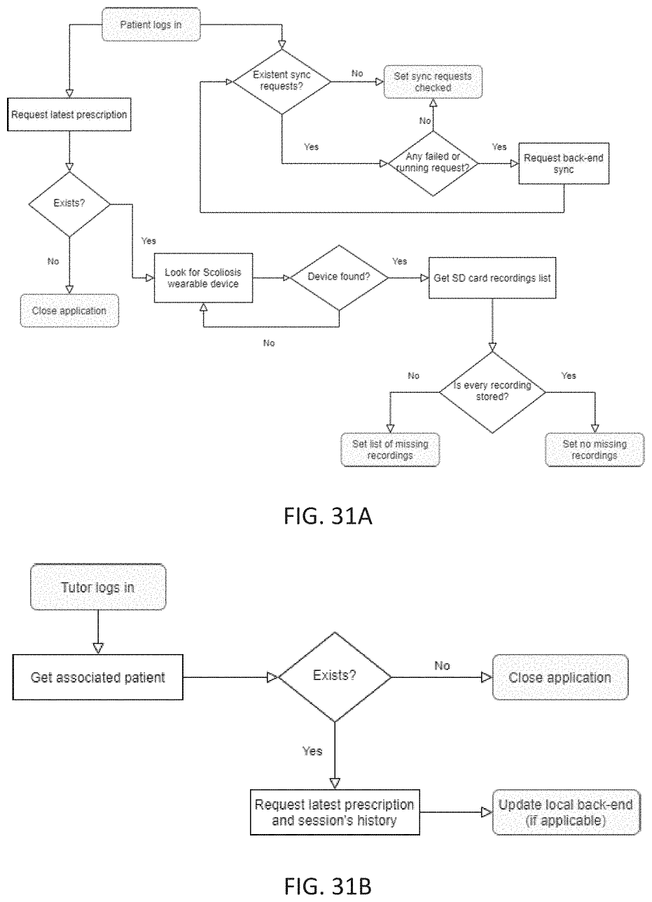

FIGS. 31A-31B show block diagrams of exemplary embodiments of workflow algorithms of a patient and of a tutor for an exemplary software application for the electronic monitor.

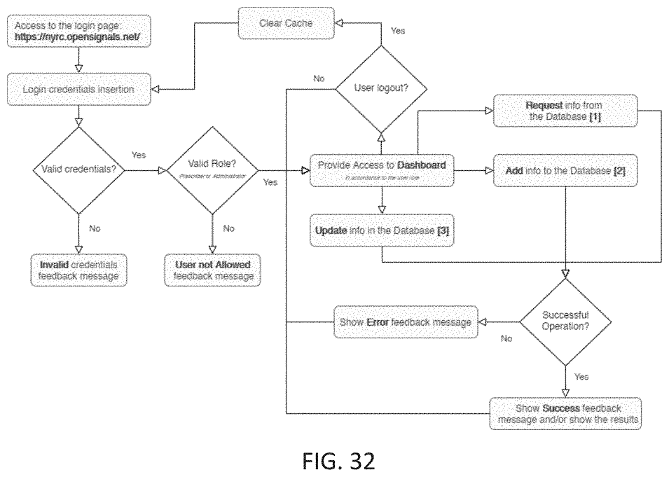

FIG. 32 shows a block diagram of an exemplary embodiment of a workflow algorithm of using an exemplary software application for the electronic monitor.

The Scoliosis Monitor is a new system that allows the continuous monitoring of the scoliosis brace use compliance and posture. This product includes a wearable brace monitor that enables the monitoring of the scoliosis brace. The wearable device is able to communicate with a companion mobile application that stores and processes the collected data and is responsible for data synchronization. All the data related with the system including data collected by the wearable device is stored in a cloud infrastructure, which can be accessed through a web-based application. Different types of user roles are defined on this system and have differentiated access to the existent data. User roles are defined as follows: A patient is a person who uses the scoliosis brace and is monitored by the brace wearable. This type of user only has access to the mobile application. A tutor is someone who is responsible for a patient and has permission to monitor his daily and past usage of the scoliosis wearable device through the mobile application. A prescriber is a person who is associated with a clinic and who is responsible for the management of the patients from this clinic. This management includes the registry of patients and the prescription of devices. Prescribers have access to all the data that is uploaded by their patients through the web application. An administrator is a person who has any and all the privileges of all the other types of users. They have the authority to register clinics and manage all the users.

The Scoliosis Monitor is a new platform which intends to verify if the scoliosis brace is used in compliance with the goals defined by a prescriber on a prescription. The system is also intended to ensure the monitoring of a patient's posture in the long-term. The context diagram, in FIG. 28, illustrates the external entities and system interfaces. The system administrator will be able to create clinics and manage other users. A patient is always associated with a prescriber which is responsible for defining personalized goals for the scoliosis brace usage. These goals are to be monitored by the wearable device which collects data regarding the scoliosis brace usage. The collected data is to be sync upon patient log in to the Scoliosis Monitor mobile application. The data is then processed, locally stored, and sent to the remote database when possible. Access to the collected data on the mobile application is restricted to the correspondent patient and associated tutor. Users that have access to the web-based application are able to access data stored remotely.

System Components: The Scoliosis Monitor main components include: Scoliosis brace; Scoliosis wearable device; Mobile application (supporting Android and iOS operating systems); Cloud (composed of web-application, web-server and Database). Monitor device is made of a 16-bit micro-controller (32 MHz), internal battery and charging circuits, a load cell and signal conditioning circuits, Bluetooth communication module and a 16 GB SD card for internal storage. FIG. 29 shows the high-level overview of hardware main modules. The Bluetooth communication module is compatible with the Bluetooth 4.1 specification providing Bluetooth Classic communication capability and the BLE protocol. It has an integrated antenna allowing a line-of-sight range of 200 to 400 meters. In an indoor environment, stable connections should be achieved at distances of up to 6 meters. These modules allow a communication speed of 1 Mbps when using the SPP (Serial Port Profile) profile.

In FIG. 30A it is shown the design of the monitor device enclosure with the legend of the several elements of input/output from and to the user. The battery charging is performed via USB connector. When battery is charging, Charging LED presents orange color. When battery is fully charged, Charging LED presents purple color.

Device Operation: The User Button has two behaviours: Short-press (<2 sec): starts/stops SD card acquisition; Long-press (>4 sec): enables/disables BTH modem operation. The SD card acquisition is only available when internal RTC (Real Time Clock) is updated, this means that is mandatory that the device has been connected to the APP at least once. When user presses the button to start an acquisition on internal memory, devices start a session acquiring data at 1 sample per second (1 Hz). Session can be stopped by short pressing the button again. When user presses button for more than 4 seconds, devices starts advertising with “scoliosis plux XX:XX” Bluetooth friendly name (XX:XX are the last 2 digits of the MAC address). After a BTH or BLE connection is established, the device starts sending sensor data frames at 10 samples per sec (10 Hz). Table 1 summarizes all the operation states device can present and contains LED behaviour for each of the states described above.

Software Considerations: The system's software was developed based on the specifications included in the Software Requirements Specifications (SRS). It is a fully functional bundle of the mobile applications and system's Cloud that allow the prescription, recording and monitoring of the usage of the hardware system (composed of the scoliosis brace and the smartbuckle device). All software components of the system are described in detail below.

Mobile Applications: The system contains two mobile applications, for both Android and iOS operation systems. Both applications were developed natively with small adaptations to answer to the specific constraints and workflows of the operating system in which they will run. The mobile applications are the interface of the patients and tutors with the system. These applications are responsible for: Collect and processed the data recorded by the smartbuckle device; Allows the patient to train, based on the balance calculated using the smartbuckle device events; Allow the patient to monitor his usage compliance; Allow the tutor to monitor the patient's, which he is responsible for, usage compliance.

The following sections will include a description of the application's workflow, the permissions that the user must grant to use the application, the user interface, and details about data storage.

Application Workflow: In this section, it is described the several different workflows that take place during the usage of Scoliosis Monitor application. As the user opens the application, the first workflow, FIG. 30B, that takes place consists in checking if there is an account with a valid token stored. In case valid credentials exist the login screen is skipped and the user is directed to the main screen. However, it is first checked if all the necessary permissions have been granted (only needed for patients). If some permission is denied the user is then asked to grant it. If the user refuses to give the necessary permission the application closes. On the other hand, if no valid credentials are stored, the login screen is shown and to be able to access the application's content the user needs to insert and submit valid credentials.

Although there are more than two types of user roles, only patients and tutors can access Scoliosis Monitor application's content. If any other user with a different type of role tries to login the access is denied and appropriate feedback is displayed on the screen upon credentials' submission. The content displayed to tutors and patients is not exactly the same and the differences will be explained latter. As the content presented to the user differs upon its user role, different workflows take place for patients and tutors in order to manage and display the necessary data. For the next described workflows it will be mentioned several data requests. These requests may be local requests or remote requests depending on the network conditions and user settings regarding mobile data access at the time of the given request. Remote requests are always privileged, being local requests only used when the former cannot be used.

Patient specific workflows: Patient Data Setup Workflow: As a patient logs in, two different work paths start at the same time (FIG. 31A): 1. Check existing remote back-end requests: a. It is checked if there is any registry of failed remote back-end upload requests. If some exists, a sync request is done. This request includes the verification of each existing local session. It is checked if some session is yet to be sent to the remote back-end or if any remote session needs to be updated. 2. Check latest prescription and get SD card data: a. It is requested the latest user prescription. If no prescriptions are available, the application is closed. b. As soon as the latest prescription gets received the application starts to look for the Scoliosis wearable device whose MAC address is set on the given prescription. c. When the device is found, and the connection is established it is requested the list of available recordings on the device's SD card. d. Local database is then checked to find out which recordings are still not locally stored. When both described workflow paths reaches their end, a new workflow starts—the SD card reading workflow.

SD card reading workflow: Recordings existent on the Scoliosis wearable device's SD card that were set as missing are read one by one, following these steps: 1. Firstly, the recording is read from the SD card and the received data is sub-sampled and analysed. 2. General statistics and sub-sampled data are then associated with the correct day, or days, and are locally stored as a new session or an update of an existent session if that's the case. Steps 1 and 2 are repeated for each recording set as missing until there are no more missing recordings. Finally, a remote back-end sync request is done and all the local sessions that need to be uploaded or updated are sent to the cloud.

Tutor specific workflows: Tutor Data Setup Workflow: When a tutor logs in, it is necessary to get all the data that concerns its associated patient. The workflow, FIG. 31B, happens as follows: 1. Associated patient is requested. 2. As soon as the associated patient gets received it is requested both the patient's latest prescription and its session's history. 3. When the requested data is received, the local back-end is updated. Regarding session history, only the general information gets received and stored. Detailed information is requested when needed.

Data storage: Aiming to ensure a pleasing user experience while using Scoliosis Monitor application, different types of data are stored. This includes Scoliosis Monitor's user accounts, user preferences, data acquired with Scoliosis wearable device, and specific user data, such as prescriptions and other user related details. To store user preferences it is used a built-in mechanism provided by the operating systems which allows data storage in a key-value structure. There is only one preference on Scoliosis Monitor application that can be set by the user and which is the permission to use mobile data to upload or download data to and from the remote back-end. The remaining data that is stored includes: user details such as the username, sub-sampled sessions' acquisition data, and session's general statistics. All this data is stored in a local database, and can only be accessed by a logged in user and is available through the app's different screens.

Cloud: The Scoliosis Monitor system's Cloud is composed by a web-app, a web-sever and a data base. The applications ensure the control of the data collection process and, consequently, is responsible to fill the remote database with the results of the local data processing task. On the other hand, the Web-app, that will be presented in the current section, provides a simple interface where prescribers and administrators can: Visualise data produced during the training sessions (conducted in the Mobile APP); Add new patients, tutors, prescribers and administrators, in accordance to the logged in user role; Edit existing users: Enable/Disable users in order to control the access to the mobile and web platforms; Update personal data. Insert new clinics and devices into the database; Define new configuration parameters (or editing existing ones), such as: Device Types; Device Locations; Brace Types; Curve Patterns.

Application workflow: Since the web-app doesn't directly interact with the Scoliosis Monitor, following a more conventional architecture using a REST API, only a generical workflow will be provided, which can be summarized in the following items: 1. Users can access the https://nyrc.opensignals.net/ web page through a browser, providing its login credentials; 2. If the credentials are valid and the role of the user linked to them has access to the Web-app then a dashboard will be presented; 3. The content of the dashboard will depend on the role of the logged in user, i.e., an administrator will have access to more functionalities than a prescriber, considering the privileges linked to each account type; 4. Three major operations can be executed (described on Table 2); 5. The success/unsuccess of the previous requests is presented to the user.

The foregoing description discloses exemplary embodiments of the invention. While the invention herein disclosed has been described by means of specific embodiments and applications thereof, numerous modifications and variations could be made thereto by those skilled in the art without departing from the scope of the invention set forth in the claims. Modifications of the above disclosed apparatus and methods that fall within the scope of the claimed invention will be readily apparent to those of ordinary skill in the art. Accordingly, other embodiments may fall within the spirit and scope of the claimed invention, as defined by the claims that follow hereafter.

In the description above, numerous specific details are set forth in order to provide a more thorough understanding of embodiments of the invention. It will be apparent, however, to an artisan of ordinary skill that the invention may be practiced without incorporating all aspects of the specific details described herein. Not all possible embodiments of the invention are set forth verbatim herein. A multitude of combinations of aspects of the invention may be formed to create varying embodiments that fall within the scope of the claims hereafter. In addition, specific details well known to those of ordinary skill in the art have not been described in detail so as not to obscure the invention. Readers should note that although examples of the invention are set forth herein, the claims, and the full scope of any equivalents, are what define the metes and bounds of the invention protection.

### REFERENCES

The following references are cited in the Description of Related Art, and, although mentioned for historical context, none of them is considered material to the patentability of the invention as described and claimed herein.
[1]: Scoliosis. (n.d.). Retrieved Dec. 10, 2017, from http://www.aans.org/Patients/Neurosurgical-Conditions-and-Treatments/Scoliosis
[2]: Conditions and Treatments for Adult Scoliosis. (n.d.). Retrieved Jul. 5, 2019, from https://www.srs.org/patients-and-families/conditions-and-treatments/adults/scoliosis
[3]: Aebi M. The adult scoliosis. Eur Spine J. 2005; 14(10):925-948. doi: 10.1007/s00586-005-1053-9.
[4]: Zaina F, De Mauroy J C, Grivas T, Hresko M T, Kotwizki T, Maruyama T, Price N, Rigo M, Stikeleather L, Wynne J, Negrini S. Bracing for scoliosis in 2014: state of the art. Eur J Phys Rehabil Med. 2014; 50(1):93-110.
[5]: Minsk et al. Scoliosis and Spinal Disorders. 2017; 12:7DOI 10.1186/s13013-017-0117-z
[6]: Labelle H(1), Dansereau J, Bellefleur C, Poitras B. Three-dimensional effect of the Boston brace on the thoracic spine and rib cage. Spine (Phila Pa. 1976). 1996 Jan. 1; 21(1):59-64.
[7]: Rigo and Jelačić Scoliosis and Spinal Disorders. (2017) 12:10. DOI 10.1186/s13013-017-0114-2
[8]: Lebel DE(1), Al-Aubaidi Z, Shin E J, Howard A, Zeller R. Three-dimensional analysis of brace biomechanical efficacy for patients with AIS Eur Spine J. 2013 November; 22(11):2445-8. doi: 10.1007/s00586-013-2921-3. Epub 2013 Jul. 20.

We claim:

1. A scoliosis brace comprising:
a lumbar belt and
a back panel adapted to be attached to the lumbar belt,
wherein the back panel has a Y shape and includes three de-rotational pads,
wherein the Y shape has a trunk section and two branch sections,
wherein the trunk section and the two branch sections meet at a central portion,
wherein the two branch sections comprise an upper branch section and a lower branch section when the Y shape is oriented horizontally on a lateral side of the Y shape,
wherein the trunk section has a trunk section end, and one de-rotational pad is disposed at the trunk section end,
wherein each branch section has a branch section end, and one de-rotational pad is disposed at each branch section end,
wherein the Y shape is adapted to horizontally span a wearer's back from a first side of the wearer to a second side of the wearer when the wearer wears the scoliosis brace, with the trunk section adapted to support the first side and the two branch sections adapted to support the second side,
wherein each de-rotational pad of the two branch sections includes an adjustable adjustment strap that is adapted to connect to a ventral area of the lumbar belt when the lumber belt is donned,
wherein the lumbar belt includes a pull-tab cord system adapted to adjust and tighten the lumbar belt according to the wearer's physique when the lumbar belt is donned, and
wherein at least one of the sections is adapted to be adjustable as an adjustable section and is adapted to include an adjustability relative to the central portion, in which the adjustability is selected from a group consisting of:
a section length adjustment for a length-adjustable section,
a section orientation adjustment for orientation-adjustable section,
a section horizontal adjustment for a horizontally-adjustable section, and
a section vertical adjustment for a vertically-adjustable section.

2. The scoliosis brace of claim 1, wherein the adjustability comprises the section length adjustment, and the length-adjustable section comprising:
the trunk section, the upper branch section, or the lower branch section,
wherein at least one of a trunk length of the trunk section, an upper branch length of the upper branch section, and a lower branch length of the lower branch section is length-adjustable relative to the central portion,
wherein at least one length-adjustable section has an adjustable section length relative to the central portion, and
wherein the at least one length-adjustable section is adapted to have the section length adjusted using a section length adjustment mechanism.

3. The scoliosis brace of claim 1, wherein the adjustability comprises the section orientation adjustment, and the orientation-adjustable section comprising:
the trunk section, the upper branch section, or the lower branch section,
wherein at least one of a trunk orientation of the trunk section, an upper branch section orientation of the upper branch section, and a lower branch section orientation of the lower branch section is orientation-adjustable relative to the central portion,
wherein at least one orientation-adjustable section has an adjustable section orientation relative to the central portion,
wherein the central portion includes at least one orientation adjustment mechanism, and
wherein the at least one orientation-adjustable section is adapted to have the section orientation adjusted using a section orientation adjustment mechanism, in which the at least one orientation adjustment mechanism comprises the section orientation adjustment mechanism.

4. The scoliosis brace of claim 1, wherein the adjustability comprises the section horizontal adjustment, and the horizontally-adjustable section comprising:
the trunk section, the upper branch section, or the lower branch section,
wherein at least one of a trunk horizontal position of the trunk section, an upper branch section horizontal position of the upper branch section, and a lower branch section horizontal position of the lower branch section is horizontally-adjustable relative to the central portion,
wherein at least one horizontally-adjustable section has an adjustable section horizontal position relative to the central portion, and
wherein the at least one horizontally-adjustable section is adapted to have the section horizontal position adjusted using a section horizontal position adjustment mechanism, in which the at least one orientation adjustment mechanism comprises the section orientation adjustment mechanism.

5. The scoliosis brace of claim 1, wherein the adjustability comprises the section vertical adjustment, and the vertically-adjustable section comprising:
the trunk section, the upper branch section, or the lower branch section,
wherein at least one of a trunk vertical position of the trunk section, an upper branch section vertical position of the upper branch section, and a lower branch section vertical position of the lower branch section is vertically-adjustable relative to the central portion,
wherein at least one vertically-adjustable section has an adjustable section vertical position relative to the central portion,
wherein the central portion includes at least one vertical position adjustment mechanism, and
wherein the at least one vertically-adjustable section is adapted to have the section vertical position adjusted using a section vertical position adjustment mechanism, in which the at least one vertical position adjustment mechanism comprises the section vertical position adjustment mechanism.

6. The scoliosis brace of claim 1, in which the central portion further comprises:
an adjustment mechanism present at a connection of the adjustable section and the central portion and adapted to enable the adjustability of the adjustable section relative to the central portion,
wherein the adjustment mechanism comprises at least one of a lockable gear, a tightenable winch, a threaded screw, a threaded-screw knob, a threaded bolt, a threaded nut, a pin, an axle, a rivet, a slidable bar, a lockable slot, a lockable flange, a spring-loaded engagement, a spring-biased release, a laced cord, a rubber strip, an elastic band, and an attachment hole.

7. The scoliosis brace of claim 1, wherein the back panel includes a D-shaped hole at each de-rotational pad of each branch section, and the adjustable strap of each de-rotational pad is attachable to the de-rotational pad through the D-shaped hole.

8. The scoliosis brace of claim 1, wherein the back panel includes an attachment hole proximate the central portion, and the attachment hole is adapted for use in attaching the back panel to the lumbar belt.

9. The scoliosis brace of claim 8, wherein the back panel includes a fastener on an inner face of the back panel, the fastener is affixed laterally adjacent to the attachment hole, and the fastener is adapted to receive a corresponding fastener from the lumbar belt for use in attaching the back panel to the lumbar belt.

10. The scoliosis brace of claim 9, wherein the fastener of the back panel and the corresponding fastener from the lumbar belt are adapted to be aligned and adjusted in more than one orientation when attaching the back panel to the lumbar belt, and each orientation is adapted to create a specific geometric configuration of de-rotational pads to apply specific adjustive, corrective, or supportive forces for a specific scoliosis curve of the wearer.

11. The scoliosis brace of claim 10, wherein the fastener of the back panel and the corresponding fastener from the lumbar belt are adapted to be aligned and adjusted to configure the de-rotational pads respectively as thoracic, lumbar, and pelvic pads that respectively are adapted to apply de-rotational forces at and to thoracic, lumbar, and pelvic regions of the wearer, in which said de-rotational forces are adjustable appropriately to the specific scoliosis curve of the wearer by adjusting the adjustable straps and the lumbar belt.

12. A method of providing a scoliosis brace and adjusting the scoliosis brace to a wearer, the method comprising:
providing a lumbar belt,
providing a back panel adapted to be attached to the lumbar belt,
adjusting the back panel to the wearer in adjusting an adjustability of the back panel according to needs of the wearer, and
adjusting the lumbar belt to the wearer in attaching the back panel to the lumbar belt,
wherein the back panel has a Y shape and includes three de-rotational pads,
wherein the Y shape has a trunk section and two branch sections,
wherein the trunk section and the two branch sections meet at a central portion,
wherein the two branch sections comprise an upper branch section and a lower branch section when the Y shape is oriented horizontally on a lateral side of the Y shape,
wherein the trunk section has a trunk section end, and one de-rotational pad is disposed at the trunk section end,
wherein each branch section has a branch section end, and one de-rotational pad is disposed at each branch section end,
wherein the Y shape is adapted to horizontally span a wearer's back from a first side of the wearer to a second side of the wearer when the wearer wears the scoliosis brace, with the trunk section adapted to support the first side and the two branch sections adapted to support the second side,
wherein each de-rotational pad of the two branch sections includes an adjustable adjustment strap that is adapted to connect to a ventral area of the lumbar belt when the lumber belt is donned,
wherein the lumbar belt includes a pull-tab cord system adapted to adjust and tighten the lumbar belt according to the wearer's physique when the lumbar belt is donned, and
wherein at least one of the sections is adapted to be adjustable as an adjustable section and is adapted to include the adjustability relative to the central portion, in which the adjustability is selected from a group consisting of:
a section length adjustment for a length-adjustable section,
a section orientation adjustment for orientation-adjustable section,
a section horizontal adjustment for a horizontally-adjustable section, and
a section vertical adjustment for a vertically-adjustable section.

13. The method of claim 12, wherein the adjustability comprises the section length adjustment, and the length-adjustable section comprising:
the trunk section, the upper branch section, or the lower branch section,
wherein at least one of a trunk length of the trunk section, an upper branch length of the upper branch section, and a lower branch length of the lower branch section is length-adjustable relative to the central portion,
wherein at least one length-adjustable section has an adjustable section length relative to the central portion, and
wherein the at least one length-adjustable section is adapted to have the section length adjusted using a section length adjustment mechanism.

14. The method of claim 12, wherein the adjustability comprises the section orientation adjustment, and the orientation-adjustable section comprising:
the trunk section, the upper branch section, or the lower branch section,
wherein at least one of a trunk orientation of the trunk section, an upper branch section orientation of the upper branch section, and a lower branch section orientation of the lower branch section is orientation-adjustable relative to the central portion,
wherein at least one orientation-adjustable section has an adjustable section orientation relative to the central portion,
wherein the central portion includes at least one orientation adjustment mechanism, and
wherein the at least one orientation-adjustable section is adapted to have the section orientation adjusted using a section orientation adjustment mechanism, in which the at least one orientation adjustment mechanism comprises the section orientation adjustment mechanism.

15. The method of claim 12, wherein the adjustability comprises the section horizontal adjustment, and the horizontally-adjustable section comprising:
the trunk section, the upper branch section, or the lower branch section,
wherein at least one of a trunk horizontal position of the trunk section, an upper branch section horizontal position of the upper branch section, and a lower branch section horizontal position of the lower branch section is horizontally-adjustable relative to the central portion,
wherein at least one horizontally-adjustable section has an adjustable section horizontal position relative to the central portion, and
wherein the at least one horizontally-adjustable section is adapted to have the section horizontal position adjusted using a section horizontal position adjustment mechanism, in which the at least one orientation adjustment mechanism comprises the section orientation adjustment mechanism.

16. The method of claim 12, wherein the adjustability comprises the section vertical adjustment, and the vertically-adjustable section comprising:
the trunk section, the upper branch section, or the lower branch section,
wherein at least one of a trunk vertical position of the trunk section, an upper branch section vertical position of the upper branch section, and a lower branch section vertical position of the lower branch section is vertically-adjustable relative to the central portion,
wherein at least one vertically-adjustable section has an adjustable section vertical position relative to the central portion,
wherein the central portion includes at least one vertical position adjustment mechanism, and
wherein the at least one vertically-adjustable section is adapted to have the section vertical position adjusted using a section vertical position adjustment mechanism, in which the at least one vertical position adjustment mechanism comprises the section vertical position adjustment mechanism.

17. The method of claim 12, in which the central portion further comprises:
an adjustment mechanism present at a connection of the adjustable section and the central portion and adapted to enable the adjustability of the adjustable section relative to the central portion,
wherein the adjustment mechanism comprises at least one of a lockable gear, a tightenable winch, a threaded screw, a threaded-screw knob, a threaded bolt, a threaded nut, a pin, an axle, a rivet, a slidable bar, a lockable slot, a lockable flange, a spring-loaded engagement, a spring-biased release, a laced cord, a rubber strip, an elastic band, and an attachment hole.

18. The method of claim 12, wherein the adjusting the back panel to the wearer comprises:
aligning and configuring the sections and adjusting each adjustable section so that the de-rotational pads respectively are aligned, adjusted, and configured relative to the wearer to be thoracic, lumbar, and pelvic pads that respectively are adapted to apply de-rotational forces at and to thoracic, lumbar, and pelvic regions of the wearer, in which said de-rotational forces are adjustable appropriately to the specific scoliosis curve of the wearer.

19. A scoliosis brace back panel for use with a scoliosis brace, the back panel comprising:
an assembly of three back piece sections and a central portion, the central portion being adapted to be adjustably attached to a lumbar belt;
wherein the back panel has a Y shape and includes three de-rotational pads,
wherein the Y shape has a trunk section and two branch sections,
wherein the trunk section and the two branch sections meet at the central portion,
wherein the two branch sections comprise an upper branch section and a lower branch section when the Y shape is oriented horizontally on a lateral side of the Y shape,
wherein the trunk section has a trunk section end, and one de-rotational pad is disposed at the trunk section end,
wherein each branch section has a branch section end, and one de-rotational pad is disposed at each branch section end,
wherein the Y shape is adapted to horizontally span a wearer's back from a first side of the wearer to a second side of the wearer when the wearer wears the scoliosis brace, with the trunk section adapted to support the first side and the two branch sections adapted to support the second side,
wherein each de-rotational pad of the two branch sections includes an adjustable adjustment strap that is adapted to connect to a ventral area of the lumbar belt when the lumber belt is donned, and
wherein at least one of the sections is adapted to be adjustable as an adjustable section and is adapted to include an adjustability relative to the central portion, in which the adjustability is selected from a group consisting of:
a section length adjustment for a length-adjustable section,
a section orientation adjustment for orientation-adjustable section,
a section horizontal adjustment for a horizontally-adjustable section, and
a section vertical adjustment for a vertically-adjustable section.

20. The scoliosis brace back panel of claim 19:
wherein the assembly includes an intersection of the trunk section and the two branch sections, and the intersection is adapted to be positioned proximate a center of the wearer's back when the wearer wears the brace;
wherein the assembly includes a space located vertically between the two branch sections and formed by the two branch sections separating and extending from the intersection with the trunk section, and the space is adapted to be positioned proximate the first side of the wearer or the second side of the wearer when the wearer wears the brace;
wherein each branch section includes a D-shaped hole at each de-rotational pad of the branch section, and the adjustable strap of each de-rotational pad is adapted to be attached to the de-rotational pad through the D-shaped hole;
wherein the assembly includes an attachment hole proximate the intersection of the trunk section and the two branch sections, and the attachment hole is adapted for use in attaching the back panel to the lumbar belt; and
wherein the assembly includes a fastener on an inner face of the central portion, the fastener is affixed laterally adjacent the attachment hole, and the fastener is adapted to receive a corresponding fastener from the lumbar belt for use in attaching the back panel to the lumbar belt.
